(19) United States
(12) Patent Application Publication
(10) Pub. No.: US 2025/0079020 A1
(43) Pub. Date: Mar. 6, 2025

(54) METHOD OF AND SYSTEM FOR TRAINING AND USING MACHINE LEARNING MODELS FOR PRE-INTERVENTIONAL PLANNING AND POST-INTERVENTIONAL MONITORING OF ENDOVASCULAR AORTIC REPAIR (EVAR)

(71) Applicant: ViTAA MEDICAL SOLUTIONS INC., Montreal (CA)

(72) Inventors: Arianna FORNERIS, Calgary (CA); Elena DI MARTINO, Calgary (CA); Atefeh ABDOLMANAFI, Keswick (CA)

(73) Assignee: ViTAA MEDICAL SOLUTIONS INC., Montreal, QC (CA)

(21) Appl. No.: 18/711,280

(22) PCT Filed: Nov. 18, 2022
(86) PCT No.: PCT/IB2022/061152

§ 371 Date: May 17, 2024

Related U.S. Application Data
(60) Provisional application No. 63/264,294, filed on Nov. 19, 2021.

(51) Int. Cl. G16H 50/50; A61B 34/10

(52) U.S. Cl. CPC G16H 50/50; A61B 34/10

(57) ABSTRACT

There are provided methods and systems for training and using machine learning (ML) models to determine suitability of a given patient for an endovascular aortic repair (EVAR) intervention and to monitor the given patient after the EVAR intervention. The pre-EVAR ML models are trained on training datasets comprising strain maps having been generated based on a multiphase image stack of the aorta of each respective patient during a cardiac cycle, measures of aortic weakness in the aorta of each respective patient, geometric features of the aorta of the respective patient, and outcomes of the EVAR intervention on the respective patient. The post-EVAR ML models are trained on pre-EVAR strain maps, post-EVAR strain maps having been generated after the EVAR intervention, and a respective outcome of the EVAR intervention.

### CROSS-REFERENCE TO RELATED APPLICATIONS

The present application claims priority to U.S. Provisional Patent Application Ser. No. 63/264,294 filed on Nov. 19, 2021.

### FIELD

The present technology pertains to the field of medical imaging. More specifically, the present technology relates to a method of and a system for training and using machine learning models for pre-interventional planning and post-interventional monitoring of endovascular aortic repair (EVAR).

### BACKGROUND

Aortic aneurysms (AAs) are the end point of a multifactorial process that induces pathological and degenerative remodeling of the aortic wall (loss of elastin, inflammation, reduced load-bearing collagen) causing progressive weakening and permanent localized dilation of the artery. Abdominal aortic aneurysm (AAA) has a prevalence of 4-8% in screened populations and mainly affects the male adult population [1]. Aneurysms can expand at different rates and are usually asymptomatic until they manifest with severe hemorrhage due to rupture, characterized by a high mortality rate (90% for ruptured aneurysms) [2].

From a clinical point of view there is a lack of tools to accurately estimate the rupture potential for AAs when deciding whether and when to perform risky repair procedures. For decades clinicians have used the maximum diameter of the AA as a generalized proxy for its rupture potential; however, this approach has failed at accurately representing this risk leading to high patient mortality. Aneurysms can rupture at virtually any size, and more than 50% of such ruptures occur at a diameter lower than the cut-off size used as indication for elective surgical treatment [3,4]. Minimally invasive endovascular aortic repair (EVAR) procedure, using stent graft devices, provides potential advantages including reduction in mortality, morbidity, hospital stay, and discomfort. Even in the case of EVAR, however, recent data have shown that post-procedural complications, mostly due to mechanical failure of the device or endoleaks caused by a poor sealing of the graft, a leak through the graft wall or the presence of feeding collateral arteries into the sac, occur in a staggering 34% of cases [5].

Due to the advantages of a less invasive intervention, shorter hospital stays and better recovery, EVAR could provide a safe alternative to “waiting for the diameter to reach the cut-off” or performing invasive open repair surgery if better treatment planning and post-surgical monitoring were available.

### SUMMARY

It is an object of the present technology to ameliorate at least some of the inconveniences present in the prior art. One or more embodiments of the present technology may provide and/or broaden the scope of approaches to and/or methods of achieving the aims and objects of the present technology.

One or more embodiments of the present technology have been developed based on developers' appreciation that in the context of EVAR, stent-grafts often suffer from complications over time. Examples of such complications include but are not limited to leakage of blood or other fluids around the sealing (distal or proximal) of the devices or retrograde flow from patent adjoining vessels into the aneurysmal sac, and occlusion of the interior lumens of the device. Additionally, the materials from which vascular stent-grafts are typically made can interfere with optimal blood flow and other aspects of hemodynamics therefore affecting the overall device performance.

Due to the frequency and severity of stent-graft related complications, it is important to accurately determine whether EVAR is the right strategy for an individual patient as well as plan the device placement and monitor the patient postoperatively to prevent complications and endoleaks.

Developers of the present technology propose assessing the strength of the aortic tissue based on in vivo measurements of aortic displacement from dynamic images acquired from medical imaging devices such as, but not limited to, CT, ultrasound or MRI. This strength assessment, coupled with information on calcifications, intraluminal thrombus and aortic geometry, will give superior pre-procedural information for optimal EVAR placement as well as a suitability-based assessment of EVAR success.

For post EVAR, developers of the present technology propose assessing the displacement of the aortic wall in a 3D map, which provides an indication of pressurization of the space between the EVAR and the aortic wall, which is indicative of the presence of an endoleak.

One or more embodiments of the present technology provide for an improved pre-surgical planning and post-surgical monitoring which enable earlier and safer assessment of EVAR that can be offered to patients before complications and advanced age render surgical intervention riskier. The early identification of endoleaks and post-operative complications will change the patient's care providing a clear indication to classify patients based on risk for re-intervention. Current clinical guidelines rely on the type of endoleak (i.e., endoleak type I, II, III, IV, V) to support decision for re-intervention. However, as the level of pressurization in the aneurysmal sac is not specific to the type of endoleak, the management of the post-surgical complications remains controversial. It will be appreciated that the ability to assess individual aortas based on their specific level of deformation, as indicative of pressurization, will provide valuable information for clinical assessment and will result in an improved and patient-specific management. Additionally, post-surgical risk stratification has the potential to reduce CT imaging follow-ups frequency and, consequently, radiation exposure for patients at low risk for complications as well as overall healthcare costs.

Thus, one or more embodiments of the present technology are directed to a method of and a system for training and using a set of machine learning models for pre-interventional planning and post-interventional monitoring of Endovascular Aortic Repair (EVAR).

In accordance with a broad aspect of the present technology, there is provided a method for training a machine learning (ML) model to determine suitability of a given patient for an endovascular aortic repair (EVAR) intervention, said method being executed by at least one processor, said method comprising: receiving a training dataset, the training dataset comprises, for each patient of a set of patients having undergone an EVAR intervention: a respective strain map having been generated based on a multiphase image stack of the aorta of the respective patient during a cardiac cycle, respective measures of aortic weakness in the aorta of the respective patient, geometric features of the aorta of the respective patient, and a respective outcome of the EVAR intervention on the respective patient, receiving a pre-EVAR ML model, training the pre-EVAR ML model on the training dataset to determine suitability for an EVAR intervention by using the respective outcome as a target, said training comprises, for a respective patient: generating, using the pre-EVAR ML model, a set of features based on the respective strain map, the respective measures of aortic weakness in the aorta of the respective patient and the geometric features of the aorta of the respective patient, determining, using the pre-EVAR ML model, based on the set of features, an outcome prediction, and updating, based on the outcome prediction and the respective outcome, at least a portion of the pre-EVAR ML model to obtain an updated portion, and outputting the trained pre-EVAR ML model, the trained pre-EVAR ML model comprises at least the updated portion.

In one or more embodiments of the method, the training dataset further comprises, for each patient of the set of patients having undergone the EVAR intervention, at least one of: a respective calcification distribution map and an intraluminal thrombus thickness (ILT) map, and said generating the set of features comprises generating features from the at least one of the respective calcification distribution map and the ILT map.

In one or more embodiments of the method, the respective outcome comprises one of: a positive outcome and a negative outcome.

In one or more embodiments of the method, the respective strain map comprises a strain heterogeneity map and a strain and relative deformation map.

In one or more embodiments of the method, the set of features comprises features indicative of: heterogeneous strain at proximal and distal sealing regions of an aneurysm, and a deformation level at the neck.

In one or more embodiments of the method, the geometric features comprise at least one of: an aortic neck angle, a tortuosity of the lumen centerline, an asymmetry of an aneurysmal sac, and a deformation value at the neck.

In one or more embodiments of the method, the respective measures of aortic weakness in the aorta are in the form of a respective regional aortic weakness (RAW) map of the aorta of the respective patient.

In one or more embodiments of the method, the training dataset further comprises, for each patient of the set of patients having undergone the EVAR intervention: respective dimensions and configuration of a respective stent installed during the EVAR intervention, and said method further comprises: receiving a further ML model, training the further ML model on the training dataset to determine dimensions and configurations of a stent by using the respective dimensions and configuration of a respective stent as a target, said training comprises, for a respective patient: generating, by the further ML model, a further set of features from the respective strain map, the respective measures of aortic weakness in the aorta of the respective patient and the geometric features, determining, based on the set of features, a predicted dimension and configuration, and updating, based on the predicted dimension and configuration and the respective dimensions and configuration, at least a portion of the further ML model to obtain an updated portion, and outputting the trained further ML model, the trained further ML model comprises at least the updated portion.

In accordance with a broad aspect of the present technology, there is provided a method for training a machine learning (ML) model to monitor a given patient after an endovascular aortic repair (EVAR) intervention, the method being executed by at least one processor, said method comprises: receiving a training dataset, the training dataset comprises, for each patient of a set of patients having undergone an EVAR intervention: a respective pre-EVAR strain map having been generated based on a multiphase image stack of the aorta of the respective patient during a cardiac cycle prior to the EVAR intervention, a respective post-EVAR strain map having been generated based on a further multiphase image stack of the aorta of the respective patient during a cardiac cycle after the EVAR intervention, a respective outcome of the EVAR intervention on the respective patient, receiving a post-EVAR ML model, training the post-EVAR ML model on the training dataset to determine correct placement of the stent by using the respective outcome as a target, said training comprises, for a respective patient: generating, using the post-EVAR ML model, a set of features from the respective pre-EVAR strain map and the respective post-EVAR strain map, determining, using the post-EVAR ML model, based on the set of features, an outcome prediction, and updating, based on the outcome prediction and the respective outcome, at least a portion of the post-EVAR ML model to obtain an updated portion, and outputting the trained post-EVAR ML model, the trained post-EVAR ML model comprises at least the updated portion.

In one or more embodiments of the method, the respective outcome comprises one of a correct stent placement and an incorrect stent placement.

In one or more embodiments of the method, said determining, by the post-EVAR ML model based on the first and the second set of features, the outcome prediction comprises: identifying, by the post-EVAR ML model, a respective level of pressurization between the respective stent and a respective aortic wall, and determining, based on the respective level of pressurization being above a threshold, the outcome prediction as being an incorrect sealing.

In one or more embodiments of the method, the method further comprises determining based on the respective level of pressurization being below the threshold, the outcome prediction as being a correct sealing.

In one or more embodiments of the method, the training dataset further comprises, for each patient of the set of patients having undergone the EVAR intervention: a respective follow-up post-EVAR strain map having been generated based on a follow-up multiphase image stack of the aorta of the respective patient during a cardiac cycle after the respective post-EVAR strain map, and an indication of a respective presence of an endoleak and an endoleak type, and said method further comprises: receiving a further post-EVAR ML model, training the further post-EVAR ML model on the training dataset to identify and classify endoleaks based on the respective indication of the respective presence of the endoleak and the endoleak type, said training comprises, for a respective patient: generating, by the further post-EVAR ML model, a set of features from the respective pre-EVAR strain map, the respective post-EVAR strain map, and the respective follow-up post-EVAR strain map, determining, by the further post-EVAR ML model, based on the set of features, a predicted endoleak presence and a predicted endoleak type, and updating, based on the predicted endoleak presence and predicted endoleak type and the respective presence of the endoleak and the endoleak type, at least a portion of the further post-EVAR ML model to obtain an updated portion, and outputting the trained further post-EVAR ML model, the trained further post-EVAR ML model comprises at least the updated portion.

In one or more embodiments of the method, the method further comprises, after said determining, by the further post-EVAR ML model, based on the set of features, the predicted endoleak presence and the predicted endoleak type: determining a size of the aneurysm sac, and determining, based on the size of the aneurysm sac and the predicted endoleak presence and the predicted endoleak type, a risk for re-intervention.

In one or more embodiments of the method, the risk for re-intervention comprises one of a low-risk and a high-risk.

In accordance with a broad aspect of the present technology, there is provided a system for training a machine learning (ML) model to determine suitability of a given patient for an endovascular aortic repair (EVAR) intervention, said system comprises: at least one processor, and a non-transitory storage medium operatively connected to the at least one processor, the non-transitory storage medium storing instructions, the at least one processor, upon executing the instructions, being configured for: receiving a training dataset, the training dataset comprises, for each patient of a set of patients having undergone an EVAR intervention: a respective strain map having been generated based on a multiphase image stack of the aorta of the respective patient during a cardiac cycle, respective measures of aortic weakness in the aorta of the respective patient, geometric features of the aorta of the respective patient, and a respective outcome of the EVAR intervention on the respective patient, receiving a pre-EVAR ML model, training the pre-EVAR ML model on the training dataset to determine suitability for an EVAR intervention by using the respective outcome as a target, said training comprises, for a respective patient: generating, using the pre-EVAR ML model, a set of features based on the respective strain map, the respective measures of aortic weakness in the aorta of the respective patient and the geometric features of the aorta of the respective patient, determining, using the pre-EVAR ML model, based on the set of features, an outcome prediction, and updating, based on the outcome prediction and the respective outcome, at least a portion of the pre-EVAR ML model to obtain an updated portion, and outputting the trained pre-EVAR ML model, the trained pre-EVAR ML model comprises at least the updated portion.

In one or more embodiments of the system, the training dataset further comprises, for each patient of the set of patients having undergone the EVAR intervention, at least one of: a respective calcification distribution map and an intraluminal thrombus thickness (ILT) map, and said generating the set of features comprises generating features from the at least one of the respective calcification distribution map and the ILT map.

In one or more embodiments of the system, the respective outcome comprises one of: a positive outcome and a negative outcome.

In one or more embodiments of the system, the respective strain map comprises a strain heterogeneity map and a strain and relative deformation map.

In one or more embodiments of the system, the set of features comprises features indicative of: heterogeneous strain at proximal and distal sealing regions of an aneurysm, and a deformation level at the neck.

In one or more embodiments of the system, the geometric features comprise at least one of: an aortic neck angle, a tortuosity of the lumen centerline, an asymmetry of an aneurysmal sac, and a deformation value at the neck.

In one or more embodiments of the system, the respective measures of aortic weakness in the aorta are in the form of a respective regional aortic weakness (RAW) map of the aorta of the respective patient.

In one or more embodiments of the system, the training dataset further comprises, for each patient of the set of patients having undergone the EVAR intervention: respective dimensions and configuration of a respective stent installed during the EVAR intervention, and the at least one processor is further configured for: receiving a further ML model, training the further ML model on the training dataset to determine dimensions and configurations of a stent by using the respective dimensions and configuration of a respective stent as a target, said training comprises, for a respective patient: generating, by the further ML model, a further set of features from the respective strain map, the respective measures of aortic weakness in the aorta of the respective patient and the geometric features, determining, based on the set of features, a predicted dimension and configuration, and updating, based on the predicted dimension and configuration and the respective dimensions and configuration, at least a portion of the further ML model to obtain an updated portion, and outputting the trained further ML model, the trained further ML model comprises at least the updated portion.

In accordance with a broad aspect of the present technology, there is provided a system for training a machine learning (ML) model to monitor a given patient after an endovascular aortic repair (EVAR) intervention, said system comprises: at least one processor, and a non-transitory storage medium operatively connected to the at least one processor, the non-transitory storage medium storing instructions, the at least one processor, upon executing the instructions, being configured for: receiving a training dataset, the training dataset comprises, for each patient of a set of patients having undergone an EVAR intervention: a respective pre-EVAR strain map having been generated based on a multiphase image stack of the aorta of the respective patient during a cardiac cycle prior to the EVAR intervention, a respective post-EVAR strain map having been generated based on a further multiphase image stack of the aorta of the respective patient during a cardiac cycle after the EVAR intervention, a respective outcome of the EVAR intervention on the respective patient, receiving a post-EVAR ML model, training the post-EVAR ML model on the training dataset to determine correct placement of the stent by using the respective outcome as a target, said training comprises, for a respective patient: generating, using the post-EVAR ML model, a set of features from the respective pre-EVAR strain map and the respective post-EVAR strain map, determining, using the post-EVAR ML model, based on the set of features, an outcome prediction, and updating, based on the outcome prediction and the respective outcome, at least a portion of the post-EVAR ML model to obtain an updated portion, and outputting the trained post-EVAR ML model, the trained post-EVAR ML model comprises at least the updated portion.

In one or more embodiments of the system, the respective outcome comprises one of a correct stent placement and an incorrect stent placement.

In one or more embodiments of the system, said determining, by the post-EVAR ML model based on the first and the second set of features, the outcome prediction comprises: identifying, by the post-EVAR ML model, a respective level of pressurization between the respective stent and a respective aortic wall, and determining, based on the respective level of pressurization being above a threshold, the outcome prediction as being an incorrect sealing.

In one or more embodiments of the system, the at least one processor is further configured for determining based on the respective level of pressurization being below the threshold, the outcome prediction as being a correct sealing.

In one or more embodiments of the system, the training dataset further comprises, for each patient of the set of patients having undergone the EVAR intervention: a respective follow-up post-EVAR strain map having been generated based on a follow-up multiphase image stack of the aorta of the respective patient during a cardiac cycle after the respective post-EVAR strain map, and an indication of a respective presence of an endoleak and an endoleak type, and the at least one processor is further configured for: receiving a further post-EVAR ML model, training the further post-EVAR ML model on the training dataset to identify and classify endoleaks based on the respective indication of the respective presence of the endoleak and the endoleak type, said training comprises, for a respective patient: generating, by the further post-EVAR ML model, a set of features from the respective pre-EVAR strain map, the respective post-EVAR strain map, and the respective follow-up post-EVAR strain map, determining, by the further post-EVAR ML model, based on the set of features, a predicted endoleak presence and a predicted endoleak type, and updating, based on the predicted endoleak presence and predicted endoleak type and the respective presence of the endoleak and the endoleak type, at least a portion of the further post-EVAR ML model to obtain an updated portion, and outputting the trained further post-EVAR ML model, the trained further post-EVAR ML model comprises at least the updated portion.

In one or more embodiments of the system, the at least one processor is further configured for, after said determining, by the further post-EVAR ML model, based on the set of features, the predicted endoleak presence and the predicted endoleak type: determining a size of the aneurysm sac, and determining, based on the size of the aneurysm sac and the predicted endoleak presence and the predicted endoleak type, a risk for re-intervention.

In one or more embodiments of the system, the risk for re-intervention comprises one of a low-risk and a high-risk.

### Terms and Definitions

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression “a server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, unless expressly provided otherwise, a computer system may refer, but is not limited to, an “electronic device”, a “client device”, a “computing device”, an “operation system”, a “system”, a “computer-based system”, a “computer system”, a “network system”, a “network device”, a “controller unit”, a “monitoring device”, a “control device”, a “server”, and/or any combination thereof appropriate to the relevant task at hand.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e. its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the servers, nor is their use (by itself) intended to imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description and the accompanying drawings.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 illustrates a schematic diagram of an electronic device in accordance with one or more non-limiting embodiments of the present technology.

FIG. 2 illustrates a schematic diagram of a communication system in accordance with one or more non-limiting embodiments of the present technology.

FIG. 3 illustrates a schematic diagram of an EVAR planning and monitoring procedure in accordance with one or more non-limiting embodiments of the present technology.

FIG. 4 illustrates a pre-surgical EVAR planning procedure in accordance with one or more non-limiting embodiments of the present technology.

FIG. 5 illustrates a post-surgical EVAR monitoring procedure in accordance with one or more non-limiting embodiments of the present technology.

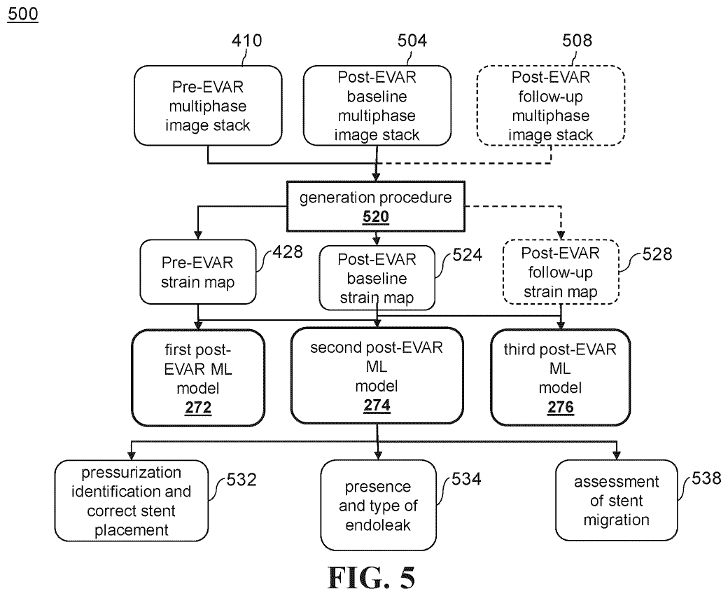

FIG. 6 illustrates inputs and outputs of a pre-surgical EVAR planning model training procedure in accordance with one or more non-limiting embodiments of the present technology.

FIG. 7 illustrates inputs and outputs of a post-surgical EVAR monitoring model training procedure in accordance with one or more non-limiting embodiments of the present technology.

FIG. 8A and FIG. 8B illustrate a computational tomography (CT) image of an implanted stent-graft of a patient having undergone an endovascular aortic aneurysm repair (EVAR) intervention having been taken after the intervention (baseline) and 10 month after the intervention (follow-up), respectively.

FIG. 9A and FIG. 9B illustrate respectively non-limiting examples of a pre-surgical strain map and a post-surgical strain map, the post-surgical strain map showing persistent post-EVAR elevated strain for a patient with persistent sac diameter but no reported endoleak.

FIG. 10 illustrates an axial view of a body of a patient acquired using a CT scan, the axial view showing the location of a small previously unrecognized Type 2 lumbar endoleak identified after repeated review of the CT images prompted by the strain map assessment.

FIG. 11A and FIG. 11 B illustrate respectively non-limiting examples of a pre-surgical strain map and pre-surgical sectional RAW map showing low, homogenous strain and low RAW index in the aortic neck region.

FIG. 12 illustrates a flowchart of a method for training a machine learning model to determine suitability of a given patient for an endovascular aortic repair (EVAR) intervention, the method being executed in accordance with one or more non-limiting embodiments of the present technology.

FIG. 13 illustrates a flowchart of a method for training a machine learning (ML) model to monitor a given patient after an endovascular aortic repair (EVAR) intervention, the method being executed in accordance with one or more non-limiting embodiments of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some non-limiting embodiments of the present technology, the processor may be a general-purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, we will now consider some non-limiting examples to illustrate various implementations of aspects of the present technology.

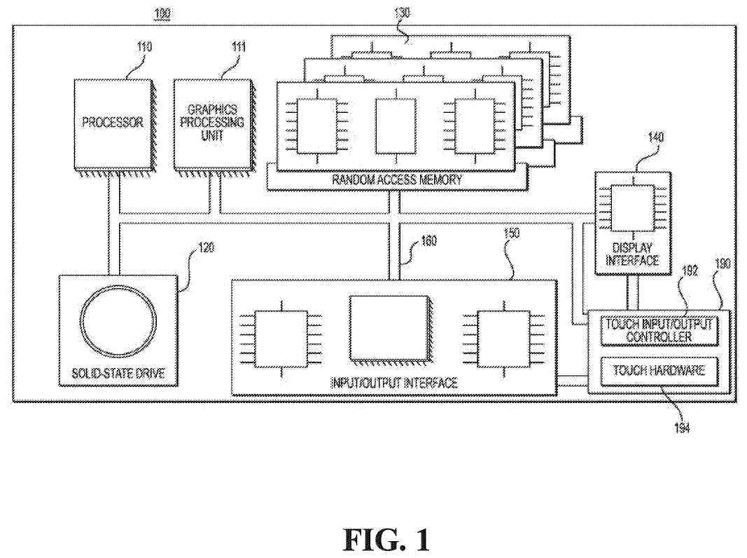

With reference to FIG. 1, there is illustrated a schematic diagram of an electronic device 100 suitable for use with some non-limiting embodiments of the present technology.

### Electronic Device

The electronic device 100 comprises various hardware components including one or more single or multi-core processors collectively represented by processor 110, a graphics processing unit (GPU) 111, a solid-state drive 120, a random-access memory 130, a display interface 140, and an input/output interface 150.

Communication between the various components of the electronic device 100 may be enabled by one or more internal and/or external buses 160 (e.g. a PCI bus, universal serial bus, IEEE 1394 “Firewire” bus, SCSI bus, Serial-ATA bus, etc.), to which the various hardware components are electronically coupled.

The input/output interface 150 may be coupled to a touchscreen 190 and/or to the one or more internal and/or external buses 160. The touchscreen 190 may be part of the display. In some embodiments, the touchscreen 190 is the display. The touchscreen 190 may equally be referred to as a screen 190. In the embodiments illustrated in FIG. 1, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the electronic device 100 in addition or in replacement of the touchscreen 190.

According to implementations of the present technology, the solid-state drive 120 stores program instructions suitable for being loaded into the random-access memory 130 and executed by the processor 110 and/or the GPU 111 for training and using machine learning models for pre-interventional planning and post-interventional monitoring of endovascular aortic repair (EVAR). For example, the program instructions may be part of a library or an application.

The electronic device 100 may be implemented in the form of a server, a desktop computer, a laptop computer, a tablet, a smartphone, a personal digital assistant or any device that may be configured to implement the present technology, as it may be understood by a person skilled in the art.

### System

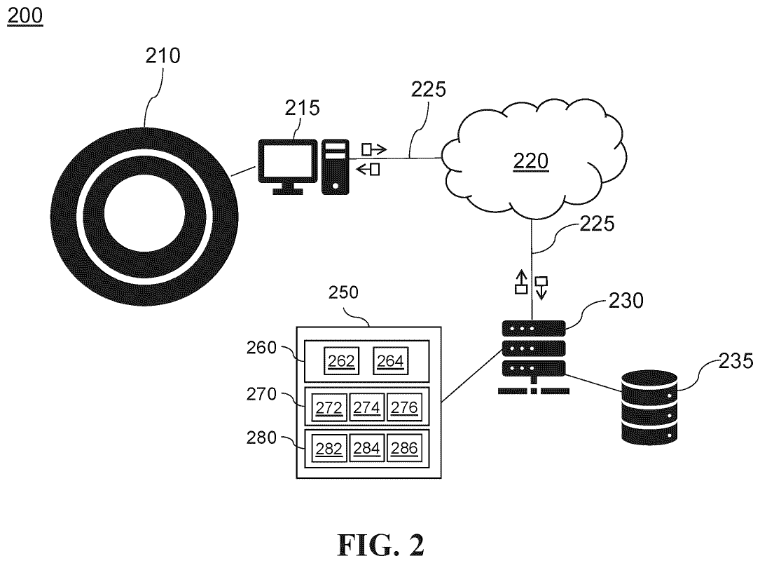

Referring to FIG. 2, there is shown a schematic diagram of a communication system 200 being suitable for implementing non-limiting embodiments of the present technology. It is to be expressly understood that the communication system 200 as illustrated is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the communication system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition it is to be understood that the communication system 200 may provide in certain instances simple implementations of the present technology, and that where such is the case they have been presented in this manner as an aid to understanding. As persons skilled in the art would understand, various implementations of the present technology may be of a greater complexity.

The communication system 200 comprises inter alia a medical imaging apparatus 210 associated with a workstation computer 215, a server 230 and a database 235 coupled over a communications network 220 via respective communication links 225 (not separately numbered).

In one or more embodiments, at least a portion of the system 200 implements the Picture Archiving and Communication System (PACS) technology.

### Medical Imaging Apparatus

The medical imaging apparatus 210 is configured to inter alia: (i) acquire, according to acquisition parameters, one or more images comprising an aorta of a given subject; and (ii) transmit the images to the workstation computer 215.

The medical imaging apparatus 210 may comprise one of: a computed tomography (CT) scanner, a magnetic resonance imaging (MRI) scanner, a 3D ultrasound and the like.

In some embodiments of the present technology, the medical imaging apparatus 210 may comprise a plurality of medical imaging apparatuses, such as one or more of a X-ray apparatus, a computational tomography (CT) scanner, a magnetic resonance imaging (MRI) scanner, an ultrasound (including 2D or 3D ultrasound), positron emission tomography (PET), single-photon emission computed tomography (SPECT), and the like.

The medical imaging apparatus 210 may be configured with specific acquisition parameters for acquiring images of the patient comprising the aorta of the patient. In one or more embodiments, the medical imaging apparatus 210 may acquire the images dynamically during a time period (e.g., cardiac cycle).

As a non-limiting example, in one or more embodiments where the medical imaging apparatus 210 is implemented as a CT scanner, a CT protocol comprising pre-operative retrospectively gated multidetector CT (MDCT—64-row multi-slice CT scanner) with variable dose radiation to capture the R-R interval may be used.

As another non-limiting example, in one or more embodiments where the medical imaging procedure comprises a MRI scanner, the MR protocol can comprise steady state T2 weighted fast field echo (TE=2.6 ms, TR=5.2 ms, flip angle 110 degree, fat suppression (SPIR), echo time 50 ms, maximum 25 heart phases, matrix 256×256, acquisition voxel MPS (measurement, phase and slice encoding directions) 1.56/1.56/3.00 mm and reconstruction voxel MPS 0.78/0.78/1.5), or similar cine acquisition of the portion of aorta under study, axial slices.

The medical imaging apparatus 210 includes or is connected to a workstation computer 215 for inter alia control of acquisition parameters and image data transmission.

In one or more embodiments, the medical imaging apparatus 210 is part of a Picture Archiving and Communication System (PACS) for storing and retrieving medical images together with the workstation computer 215 and other electronic devices such as the server 230.

### Workstation Computer

The workstation computer 215 is configured to inter alia: (i) control parameters of the medical imaging apparatus 210 and cause acquisition of images; and (ii) receive and process the plurality of images from the medical imaging apparatus 210.

In one or more embodiments. the workstation computer 215 may receive images in raw format and perform a tomographic reconstruction using known algorithms and software.

The implementation of the workstation computer 215 is known in the art. The workstation computer 215 may be implemented as the electronic device 100 or comprise components thereof, such as the processor 110, the graphics processing unit (GPU) 111, the solid-state drive 120, the random-access memory 130, the display interface 140, and the input/output interface 150.

In one or more other embodiments, the workstation computer 215 may be integrated at least in part into the medical imaging apparatus 210.

In one or more embodiments, the workstation computer 215 is configured according to the Digital Imaging and Communications in Medicine (DICOM) standard for communication and management of medical imaging information and related data.

In one or more embodiments, the workstation computer 215 may store the images in a local database (not illustrated).

The workstation computer 215 is connected to a server 230 over the communications network 220 via a respective communication link 225. In one or more embodiments, the workstation computer 215 may transmit the images and/or multiphase stack to the server 230 and/or the database 235 for storage and/or processing thereof.

### Server

The server 230 is configured to inter alia: (i) receive images having been acquired by the medical imaging apparatus 210; (ii) access a set of machine learning (ML) models 250 and training datasets; (iii) train the set of ML models 250 on the training datasets; (iv) receive or generate one or more of strain maps, respective regional aortic weakness (RAW) maps, calcification distribution maps, intraluminal thrombus (ILT) maps, and aortic neck angles; (v) determine, by using one or more of the set of ML models 250, based on the one or more of strain maps, RAW maps, calcification distribution maps, ILT maps and aortic neck angles, suitability of a given patient for an EVAR intervention and dimensions and configuration of a stent for the EVAR intervention; and (vi) monitor, by using one or more of the set of ML models 250, based on strain maps acquired after the EVAR intervention, the patient by identifying intra-sac pressurization as indicative of the presence of an endoleak/endotension, type of endoleak, identification of a low or high risk of re-intervention, and early assessment of stent-graft migration

How the server 230 is configured to do so will be explained in more detail herein below.

The server 230 can be implemented as a conventional computer server and may comprise some or all of the components of the electronic device 100 illustrated in FIG. 2. In an example of one or more embodiments of the present technology, the server 230 can be implemented as a Dell™ PowerEdge™ Server running the Microsoft™ Windows Server™ operating system. Needless to say, the server 230 can be implemented in any other suitable hardware and/or software and/or firmware or a combination thereof. In the illustrated non-limiting embodiment of present technology, the server 230 is a single server. In alternative non-limiting embodiments of the present technology, the functionality of the server 230 may be distributed and may be implemented via multiple servers (not illustrated).

The implementation of the server 230 is well known to the person skilled in the art of the present technology. However, briefly speaking, the server 230 comprises a communication interface (not illustrated) structured and configured to communicate with various entities (such as the workstation computer 215, for example and other devices potentially coupled to the network 220) via the communications network 220. The server 230 further comprises at least one computer processor (e.g., a processor 110 or GPU 111 of the electronic device 100) operationally connected with the communication interface and structured and configured to execute various processes to be described herein.

In one or more embodiments, the server 230 may be implemented as the electronic device 100 or comprise components thereof, such as the processor 110, the graphics processing unit (GPU) 111, the solid-state drive 120, the random-access memory 130, the display interface 140, and the input/output interface 150.

It will be appreciated that the server 230 may provide the output of one or more processing steps to another electronic device for display, confirmation and/or troubleshooting. As anon-limiting example, the server 230 may transmit the maps (e.g., strain map, RAW map, ILT map, etc.) and results of assessments (pre-EVAR and post-EVAR) for display on a client device configured similar to the electronic device 100 such as a smart phone, tablet, and the like.

The server 230 has access to the set of ML models 250.

### Machine Learning (ML) Models

The set of ML models 250 comprises inter alia a set of pre-EVAR ML models 260, a set of post-EVAR ML models 270 and a set of segmentation ML models 280.

Each of the set of ML models 250 is parametrized by inter alia respective model parameters and respective hyperparameters.

The model parameters are configuration variables of the ML model used to perform predictions and which are estimated or learned from training data, i.e. the coefficients are chosen during learning based on an optimization strategy for outputting a prediction. The hyperparameters are configuration variables of a ML model which determine the structure of the initial ML model and how the initial model will be trained.

Thus, each ML model is generally initialized to define a ML model architecture and determine how the ML model will be trained according to the type of prediction task, the type of input, the type of training dataset, the training environment, and the like.

The respective model parameters and respective hyperparameters are initialized (i.e., parameters and hyperparameters are selected and their values are set) to obtain an initial ML model. The initial ML model may then be trained according to a selected training strategy.

In one or more embodiments, the given initial ML model including its respective model parameters and hyperparameters may be received from another computing device connected to the server 230.

It will be appreciated that the number of model parameters to initialize will depend on inter alia the type of model (i.e., classification or regression), the architecture of the model (e.g., DNN, SVM, etc.), and the model hyperparameters (e.g. a number of layers, type of layers, number of neurons in a NN).

In one or more embodiments, the hyperparameters include one or more of: a number of hidden layers and units, an optimization algorithm, a learning rate, momentum, an activation function, a minibatch size, a number of epochs, and dropout.

As a non-limiting example, for a deep neural network, initialization includes inter alia setting the number of layers, number of weights, values of the weights for each layer, and the type of activation function to obtain the initial deep neural network.

A given ML model of the set of ML models 250 may include a given feature extractor and a given classifier. The given ML model may be based on various artificial neural networks (including deep learning architectures), such as perceptron, feed forward neural network, multilayer perceptron (MLP), convolutional neural network (CNN), radial basis functional neural network, recurrent neural network (RNN), long short-term memory (LSTM), Sequence to Sequence Models (seq2seq), and autoencoder. Non-limiting examples of neural network-based models include AlexNet, VGGNet, ResNet, DenseNet, Inception, FCN, YOLO, Faster-RCNN, CornerNet, FCN, U-Net as well as variations thereof. In one or more alternative embodiments, a given ML model of the set of ML models 250 may include a feature extractor and a regression model.

### Pre-EVAR ML Models

The set of pre-EVAR ML models 260 comprises inter alia a first pre-EVAR ML model 262 and a second pre-EVAR ML model 264. After undergoing a training or learning procedure, the pre-EVAR ML models 260 are used prior to an EVAR intervention to make predictions to inform the clinician and support the decision-making process in order to improve the success and durability of the EVAR intervention through an optimized and patient-specific planning.

The first pre-EVAR ML model 262 is configured to inter alia: (i) receive one or more of: a strain map of an aorta of a given patient, a regional aortic weakness (RAW) map, geometric features of the aorta (e.g., one or more of an aortic neck angle, a tortuosity of the lumen centerline, the asymmetry of the aneurysmal sac, a deformation value at the aortic neck), an intraluminal thrombus thickness (ILT) map, and a calcification distribution map; (ii) generate, based on the one or more of: the strain map, the RAW map, the geometric features, the ILT map, and the calcification distribution map, a first set of features; and (iii) determine, based on the first set of features, suitability of the given patient for an endovascular aortic repair (EVAR) intervention.

To achieve that purpose, the first pre-EVAR ML model 262 undergoes a training procedure, which will be explained below.

In one or more embodiments, the first pre-EVAR ML model 262 may be implemented as a classification ML model. In one or more alternative embodiments, the first pre-EVAR ML model 262 may be implemented as a regression ML model.

The second pre-EVAR ML model 264 is configured to inter alia: (i) receive one or more of: a strain map of an aorta of a given patient, a regional aortic weakness (RAW) map, geometric features of the aorta (e.g. one or more of an aortic neck angle, a tortuosity of the lumen centerline, the asymmetry of the aneurysmal sac, a deformation value at the aortic neck), an intraluminal thrombus thickness (ILT) map, and a calcification distribution map; (ii) generate, based on the one or more of: the strain map, the RAW map, the geometric features, the ILT map, and the calcification distribution map, a second set of features; and (iii) determine, based on the second set of features, dimensions and configuration of a stent for the EVAR intervention.

To achieve that purpose, the second pre-EVAR ML model 264 undergoes a training procedure, as will be explained below.

In one or more embodiments, the second pre-EVAR ML model 264 may be implemented as a regression ML model.

### Post-EVAR ML Models

The set of post-EVAR ML models 270 comprises inter alia a first post-EVAR ML model 272, a second post-EVAR ML model 274, and a third post-EVAR ML model 276.

After undergoing a training or learning procedure, The post-EVAR ML models 270 are used after the EVAR intervention to monitor the patient by identifying one or more of intra-sac pressurization as indicative of the presence of an endoleak/endotension, a type of endoleak, identification of low or high risk of re-intervention, and early assessment of stent-graft migration

The first post-EVAR ML model 272 is configured to inter alia: (i) receive a pre-EVAR strain map of an aorta of a given patient and a post-EVAR strain map of the aorta of the given patient; (ii) generate, based on the pre-EVAR strain map and the post-EVAR strain map, a third set of features; and (iii) determine, based on the third set of features, an indication of a presence of pressurization. As a non-limiting example, presence of pressurization may be indicative of an incorrect stent placement or incorrect sealing.

The second post-EVAR ML model 274 is configured to inter alia: (i) receive a pre-EVAR strain map of an aorta of a given patient, a post-EVAR baseline strain map of the aorta of the given patient, and a post-EVAR follow-up strain map; (ii) generate, based on the pre-EVAR strain map, the post-EVAR baseline strain map and the post-EVAR follow-up strain map, a fourth set of features; and (iii) determine, based on the fourth set of features, a presence and type of endoleak, and a risk for re-intervention.

The third post-EVAR ML model 276 is configured to inter alia: (i) receive a pre-EVAR strain map of an aorta of a given patient, a post-EVAR baseline strain map of the aorta of the given patient, and a post-EVAR follow-up strain map; (ii) generate, based on the pre-EVAR strain map, the post-EVAR baseline strain map and the post-EVAR follow-up strain map, a fifth set of features; and (iii) assess, based on the fifth set of features, early stent migration.

It will be appreciated that “pre-EVAR” and “post-EVAR” is used to qualify machine learning models that will be trained and used respectively to perform predictions prior to an EVAR procedure and after an EVAR procedure, and does not preclude a given pre-EVAR model and a given post-EVAR model from having a similar model architecture.

### Set of Segmentation ML Models

The set of segmentation ML models 280, which may comprise one or more ML models, is configured to perform segmentation of aortas in images (i.e., classify elements or pixels having the same category with the a same label).

The set of segmentation ML models 280 comprises inter alia a first segmentation model 282, a second segmentation model 284, and a third segmentation model 286.

Each of the first segmentation model 282, the second segmentation model 284, and the third segmentation model 286 has been respectively trained to perform segmentation of images, i.e., assign an object class label for each pixel within an image. In one or more embodiments, the segmentation comprises classification of the delimited objects or regions.

In one or more alternative embodiments, at least two of the first segmentation model 282, the second segmentation model 284 and the third segmentation model 286 may be implemented as a single ML model.

Each of the first segmentation model 282, the second segmentation model 284, and the third segmentation model 286 comprises a respective feature extractor (not shown), and a respective prediction network (not shown).

The first segmentation model 282 is configured to inter alia: (i) receive an input image; (ii) extract, via the respective feature extractor, a first set of image features therefrom; and (iii) segment, via the respective prediction network, based on the first set of image features, a region of interest (ROI) and a background in the input image.

The first segmentation model 282 is trained to perform semantic segmentation of images. The first segmentation model 282 is configured to perform foreground and background segmentation, i.e. binary segmentation. In one or more other embodiments, the first segmentation model 282 may be trained to perform multi-class semantic segmentation.

In one or more embodiments, the first segmentation model 282 has an encoder-decoder architecture.

In one or more embodiments, the first segmentation model 282 is implemented as a residual network (ResNet) based fully convolution network (FCN).

In a residual network (ResNet), building blocks are stacked on top of each other and each of them is a combination of convolutional layers with kernel sizes of 1×1, 3×3, and 5×5. The output filter banks from each building block are concatenated into a single output vector that is used as the input of the next stage. 1×1 convolutions are used for dimensionality reduction. The first segmentation model 282 uses dilated convolutions which are parametrized by a dilation rate assigned to the convolutional layer(s). Dilated convolutions, by maintaining the same stride, number of parameters, and computational cost, enable the kernel to take into account a larger filed of view at each convolutional layer, in contrast with standard patch-based CNNs. The use of dilated convolutions results in denser output feature and higher segmentation performance compared to networks with standard convolutional layers. Dilated convolutions are applied by using equation (1):

$$y [ i ] = {Σ}_{k} x [ i + r \cdot k ] w ( k ) \qquad ( 1 )$$

Where i is a location in output y. The dilated convolution with dilation rate i is applied over the feature map x with kernel w.

Thus, a ResNet-based FCN architecture enables accessing strong discriminating deep features and overcoming limitations of patch-based CNNs for segmentation tasks.

Non-limiting examples of ResNet include ResNet50 (50 layers), ResNet101 (101 layers), ResNet152 (152 layers), ResNet50V2 (50 layers with batch normalization), ResNet101V2 (101 layers with batch normalization), and ResNet152V2 (152 layers with batch normalization).

In one or more alternative embodiments, the first segmentation model 282 may be implemented based on one of: AlexNet, GoogleNet, and VGG.

In one or more embodiments, the first segmentation model 282 comprises or is followed by a shallow network (e.g. a network comprising one or two hidden layers) to identify the geometric changes of the aorta.

The second segmentation model 284 is configured to inter alia: (i) receive the region of interest (ROI) comprising the aorta; (ii) extract, via the respective feature extractor, a second set of image features therefrom; and (iii) segment, via the respective prediction network, based on the second set of image features, the ROI to obtain one or more segmented lumens.

The second segmentation model 284 is trained to perform semantic segmentation of lumens in aortas. The second segmentation model 284 is configured to perform foreground and background segmentation, i.e. binary segmentation. In one or more other embodiments, the second segmentation model 284 may be trained to perform multi-class semantic segmentation.

Similarly to the first segmentation model 282, the second segmentation model 284 may be implemented as a residual network (ResNet) based fully convolution network (FCN).

The third segmentation model 286 is configured to inter alia: (i) receive remaining tissues in the aorta; (ii) extract, via the respective feature extractor, a third set of image features therefrom; and (iii) classify, via the respective prediction network, based on the third set of image features, a pathological formation caused by an aneurysm in the remaining tissues of the aorta.

In one or more embodiments, the third segmentation model 286 obtains the remaining tissues based on the segmented aorta output by the first segmentation model 282 and segmented lumen output by the second segmentation model 284.

In one or more embodiments, the third segmentation model 286 is implemented as a combination of a convolutional neural network (CNN) and a neural network. In one or more embodiments, the third segmentation model 286 comprises a CNN as a feature extractor and a feed forward neural network as a classifier. The third segmentation model 286 is configured to perform classification of pathological tissues. In one or more embodiments, the third segmentation model 286 may be trained to perform classification of calcified versus non-calcified tissues in the aortic wall and intraluminal thrombus (if present).

The set of segmentation ML models may not be necessarily used in each and every embodiment of the present technology. It will be appreciated that in some embodiments, segmentation of tissues in aortas may be performed using traditional techniques (i.e., thresholding, region-based methods, edge-based methods, watershed-based methods, clustering-based methods), other types of ML model architectures (one or more of fully convolutional networks (FCNs), convolutional models with graphical models, encoder-decoder based models, multi-scale and pyramid network based models, R-CNN based models (for instance segmentation), dilated convolutional models and DeepLab family, recurrent neural network (RNN)-based models, attention-based models, generative models and adversarial training, convolutional models with active contour models) or a combination thereof.

### Database

The database 235 is configured to inter alia: (i) store DICOM stacks; (ii) store images; (iii) store model parameters and hyperparameters of the set of ML models 250; (iv) store datasets for training, testing and validating the set of ML models 250; and (v) store predictions output by the set of ML models 250.

In one or more embodiments, the database may store Digital Imaging and Communications in Medicine (DICOM) files, including for example the DCM and DCM30 (DICOM 3.0) file extensions. Additionally or alternatively, the database 235 may store medical image files in the Tag Image File Format (TIFF), Digital Storage and Retrieval (DSR) TIFF-based format, and the Data Exchange File Format (DEFF) TIFF-based format.

In one or more embodiments, the database 235 may store ML file formats, such as .tfrecords, .csv, .npy, and .petastorm as well as the file formats used to store models, such as .pb and .pkl. The database 235 may also store well-known file formats such as, but not limited to image file formats (e.g., .png, .jpeg), video file formats (e.g., .mp4, .mkv, etc), archive file formats (e.g., .zip, .gz, .tar, .bzip2), document file formats (e.g., .docx, .pdf, .txt) or web file formats (e.g., .html).

It will be appreciated that the database 235 may store other types of data such as validation datasets (not illustrated), test datasets (not illustrated) and the like.

### Communication Network

In some embodiments of the present technology, the communications network 220 is the Internet. In alternative non-limiting embodiments, the communication network 220 can be implemented as any suitable local area network (LAN), wide area network (WAN), a private communication network or the like. It should be expressly understood that implementations for the communication network 220 are for illustration purposes only. How a communication link 225 (not separately numbered) between the workstation computer 215 and/or the server 230 and/or another electronic device (not illustrated) and the communications network 220 is implemented will depend inter alia on how each of the medical imaging apparatus 210, the workstation computer 215, and the server 230 is implemented.

The communication network 220 may be used in order to transmit data packets amongst the workstation computer 215, the server 230 and the database 235. For example, the communication network 220 may be used to transmit requests between the workstation computer 215 and the server 230.

### EVAR Planning and Monitoring Procedure

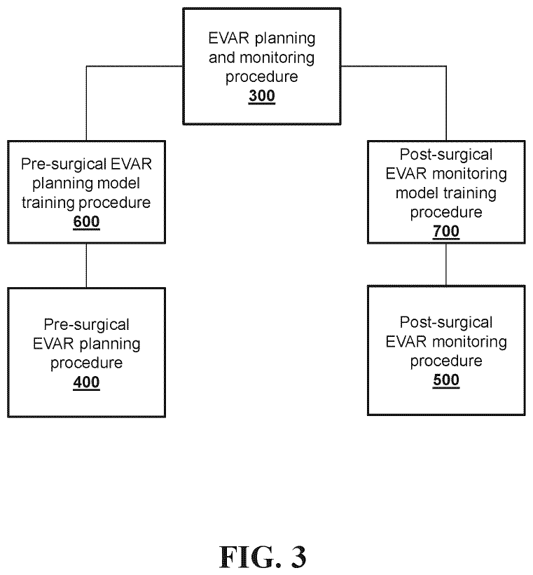

With reference to FIG. 3, there is illustrated a schematic diagram of an EVAR planning and monitoring procedure 300 in accordance with one or more non-limiting embodiments of the present technology.

The EVAR planning and monitoring procedure 300 comprises a pre-surgical EVAR planning procedure 400 and a post-surgical EVAR monitoring procedure 500.

A pre-surgical EVAR planning training procedure 600 is executed prior to the pre-surgical EVAR planning procedure 400 for training the set of pre-EVAR ML models 260.

A post-surgical EVAR monitoring model training procedure 700 is executed prior to the post-surgical EVAR monitoring procedure 500 for training the set of post-EVAR ML models 270.

In one or more embodiments of the present technology, the server 230 executes the EVAR planning and monitoring procedure 300. In one or more alternative embodiments, the server 230 may execute at least a portion of the EVAR planning and monitoring procedure 300 (i.e., at least one of the pre-surgical EVAR planning procedure 400, the post-surgical EVAR monitoring procedure 500, the pre-surgical EVAR planning training procedure 600 and post-surgical EVAR monitoring model training procedure 700), and one or more other servers (not shown) may execute other portions of the EVAR planning and monitoring procedure 300.

### Pre-Surgical EVAR Planning Procedure

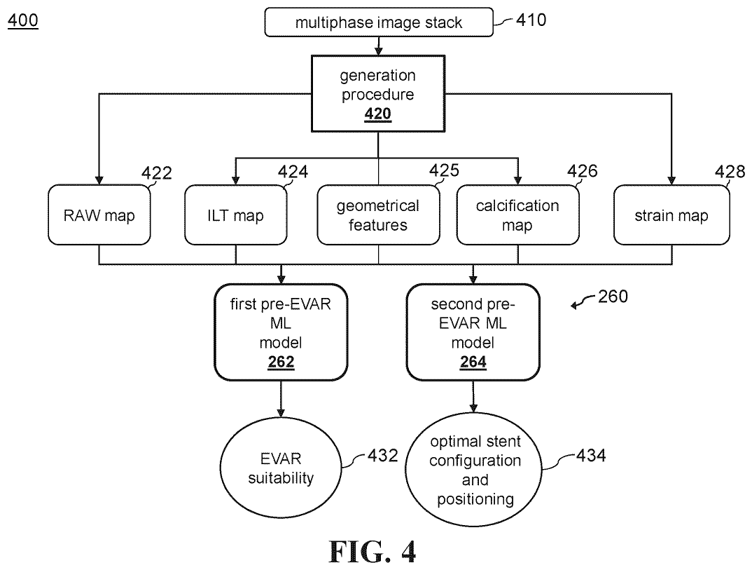

With reference to FIG. 4, the pre-surgical EVAR planning procedure 400 will now be described in accordance with one or more embodiments of the present technology.

The pre-surgical EVAR planning procedure 400 uses the set of pre-EVAR ML models 260 to determine, based on inter alia RAW maps, strain maps, ILT maps, and geometric features of the aorta (e.g. one or more of an aortic neck angle, a tortuosity of the lumen centerline, the asymmetry of the aneurysmal sac, a deformation value at the aortic neck), pre-procedural information for optimal EVAR placement including stent size and configuration, as well as a suitability-based assessment of EVAR success.

The pre-procedural information for optimal EVAR placement may then be provided as a recommendation to a clinician.

The output of the pre-surgical EVAR planning procedure 400 has the potential to inform clinician(s) and support the decision-making process in order to improve the success and durability of the EVAR intervention through an optimized and patient-specific planning.

The pre-surgical EVAR planning procedure 400 uses the set of segmentation ML models 280 to detect various aortic tissues including the wall, intraluminal thrombus (ILT), and calcification arteries in the multiphase image stack 410, and then predicts the wall strength level based on the measurements obtained from a fluid dynamic analysis.

The pre-surgical EVAR planning procedure 400 executes a generation procedure 420 to generate, based on the multiphase image stack 410, one or more of: a strain map 428 (including a strain heterogeneity map and a strain/relative deformation map), a regional aortic weakness (RAW) map 422, an ILT map 424, and a calcification distribution map 426.

It will be appreciated that the strain heterogeneity map and the strain relative deformation map are generated based on the strain map 428. The strain heterogeneity map is used to evaluate the level of heterogeneity of the strain distribution (i.e., very heterogeneous regions may affect the stent sealing and placement). The strain relative deformation map is used evaluate the strain in the aorta relative to healthier regions, so as to show areas of relative strength or weakness in order to support decision for optimal stent placement.

### Generation Procedure

The generation procedure 420 receives the multiphase image stack 410 of a given patient. The multiphase image stack 410 comprises images of an aorta of a patient having been acquired during a cardiac cycle, the patient suffering from an aortic aneurysm.

In one or more embodiments, the multiphase image stack 410 is received from the medical imaging apparatus 210 and/or the workstation computer 215.

In the context of the pre-surgical EVAR planning procedure 400, the medical images of the patient having the aortic aneurysm are acquired prior to the EVAR intervention so as to determine if an EVAR intervention is suitable, and to determine optimal stent placement and configuration.

### Strain Map

The generation procedure 420 generates, based on the multiphase stack 410, a strain map 428.

In one or more embodiments, to generate the pre-surgical strain map based on the medical images, the pre-surgical EVAR planning training procedure 600 uses the methods and systems disclosed in PCT Patent Application No. PCT/IB2020/059018 filed on Sep. 25, 2020, by the same applicant, also published as PCT Publication No. WO2021059243A1.

In one or more embodiments, the strain map 428 may be generated by another computing device based on the multiphase stack 410 and received by the generation procedure 420.

### Strain/Relative Deformation Map

In one or more embodiments, the generation procedure 420 generates, based on the strain map 428, a strain and relative deformation map.

The strain and relative deformation map comprise measures of local deformation of the aortic wall during the cardiac cycle.

The strain relative deformation map is used evaluate the strain in the aorta relative to “healthier” regions, to show areas of relative strength or weakness in order to support decision for optimal stent placement.

In one or more embodiments, the strain and relative deformation map may be generated by another computing device based on the strain map 428 and the multiphase stack 410 and may be received by the generation procedure 420.

### Strain Heterogeneity Map

In one or more embodiments, the generation procedure 420 generates, based on the strain map 428, a strain heterogeneity map.

The strain heterogeneity map is indicative of a level of heterogeneity in strain distribution in the aorta, and shows regions where strain varies compared to other regions, and where the regions may be determined based on different strain ranges.

The strain heterogeneity map is used to evaluate the level of heterogeneity of the strain distribution (i.e., very heterogeneous regions may affect the stent sealing and placement).

In one or more embodiments, the strain heterogeneity map may be generated by another computing device based on the strain map 428 and the multiphase stack 410 and may be received by the generation procedure 420.

### Regional Aortic Weakness (RAW) Map

The generation procedure 420 generates, based on the multiphase image stack 410, a pre-surgical RAW map 422.

Regional weakening (RW) analysis, which may also be referred to as regional rupture potential (RRP) analysis, enables performing assessment of vessels based on parameters that correlate with the local weakening, expansion and rupture of the vessel and provides a rationale for clinical decisions by performing calculations solely based on images acquired by a medical imaging apparatus. For an aorta, regional weakening is referred to as regional aortic weakening (RAW), which identifies areas of weakness of the wall of the aorta, including aortic aneurysms, in order to predict growth and potential rupture. A RAW index or parameter may be determined based on: region-averaged time-averaged wall shear stress (TAWSS) obtained from computational fluid dynamic (CFD) simulations, region averaged ILT thickness, and region-averaged maximum principal strain.

In one or more embodiments, to generate the pre-surgical RAW map 422 based on the medical images, the generation procedure 420 uses the methods and systems disclosed in PCT Patent Application No. PCT/IB2020/059018 filed on Sep. 25, 2020, by the same applicant, also published as PCT Publication No. WO2021059243A1.

In one or more embodiments, the generation procedure 420 determines measures of aortic weakness in the aorta of the patient without generating a complete pre-surgical RAW map 422.

The generation procedure 420 determines, for the given patient, based on the pre-surgical RAW map 422, aortic wall weakness values at the sealing zones.

In one or more embodiments, the pre-surgical RAW map 422 may be generated by another computing device based on at least the strain map 428 and the multiphase stack 410 and may be received by the generation procedure 420.

In one or more alternative embodiments, the pre-surgical RAW map 422 may be optional.

### Calcification Distribution Map and Intraluminal Thrombus Thickness Map

The generation procedure 420 accesses the set segmentation ML models 280. The segmentation ML models 280 are used to segment the aorta, iliac arteries, the lumen, and remaining tissues including wall and intraluminal thrombus (ILT) in the medical images of each patient.

In one or more embodiments, to segment the aorta, iliac arteries, the lumen, and remaining tissues including wall and intraluminal thrombus (ILT) in medical images, the generation procedure 420 uses the methods and systems disclosed in U.S. Provisional Patent Application Ser. No. 63/152,105 filed on Feb. 22, 2021, and published as PCT Application Publication no. WO 2022175924A1 by the same applicant

The generation procedure 420 generates, using the set of segmentation ML models 280, based on the multiphase stack, an intraluminal thrombus thickness (ILT) map 424 and calcification distribution map 426.

In one or more alternative embodiments, the aortic wall weakness values at the sealing zones may be obtained without using the pre-surgical RAW map. As a non-limiting example, in embodiments where the pre-surgical RAW map is not used, the aortic wall weakness values at the sealing zones may be received from another electronic device.

In one or more alternative embodiments, the generation procedure 420 determines, for each patient, a set of geometric features of the aorta. The set of geometric features includes one or more of: an aortic neck angle (i.e., landing site with respect to the aneurysmal dilatation), a tortuosity of the lumen centerline, the asymmetry of the aneurysmal sac, a deformation value at the aortic neck. In one or more alternative embodiments, the generation procedure 420 may receive one or more of the geometric features from another electronic device.

In some embodiments, the geometric features may be determined by one of the set of pre-EVAR ML models 260, where the one of the set of pre-EVAR ML models 260 is configured to extract geometric features of aortas. In one or more other embodiments, the geometric features may be extracted using manual techniques, automatic techniques or a combination thereof.

In one or more embodiments, the deformation value at the aortic neck is determined based on the strain map 428 u the multiphase image stack 410.

In one or more other embodiments, one or more of the regional aortic weakness (RAW) map, the strain heterogeneity map, the strain/relative deformation map, the intraluminal thrombus thickness (ILT) map and the calcification distribution map may be generated by one or more other electronic devices executing the generation procedure 420 and then received by the pre-surgical EVAR planning training procedure 600.

The set of pre-EVAR ML models 260 receives one or more of: the strain map 428, the strain heterogeneity map and the strain/relative deformation map, the regional aortic weakness (RAW) map 422, the ILT map 424 and the calcification distribution map 426, as well as the geometric features of the aorta.

The first pre-EVAR ML model 262 is configured to inter alia: (i) analyze one or more of the strain map 428, the strain heterogeneity map and the strain/relative deformation map, the regional aortic weakness (RAW) map 422, the ILT map 424, the calcification distribution map 426, and geometric features to extract a set of features therefrom; and (ii) determine, based on the set of features, if an EVAR intervention would be suitable for the given patient.

In one or more embodiments, the first pre-EVAR ML model 262 outputs a binary value indicative of the EVAR intervention being suitable or not for the given patient.

In one or more other embodiments, the first pre-EVAR ML model 262 may provide a score indicative of the EVAR intervention being suitable for the given patient.

In one or more embodiments, features indicative of an EVAR intervention not being suitable include: excessive deformation at the neck (which would render the sealing ineffective), highly heterogeneous strain at the level of proximal and distal sealing region of the aneurysm, presence of calcifications at the sealing/landing site, and presence of intraluminal thrombus at the sealing/landing site.

In some embodiments, in instances where the first pre-EVAR ML model 262 has determined that the given patient requires an EVAR intervention, the second pre-EVAR ML model 264 is configured to inter alia: (i) analyze one or more of the strain map 428, the strain heterogeneity map and the strain/relative deformation map, the regional aortic weakness (RAW) map 422, the ILT map 424, the calcification distribution map 426, and geometric features 425 (e.g. one or more of an aortic neck angle, a tortuosity of the lumen centerline, the asymmetry of the aneurysmal sac, a deformation value at the aortic neck) to extract a second set of features therefrom; and (ii) determine, based on the second set of features, an optimal stent configuration and placement for a reduced likelihood of post-procedural complications. It will be appreciated that in some embodiments, the optimal stent configuration and placement may be determined by a medical expert.

In one or more embodiments, one or more of the set of pre-EVAR ML models 260 may extract as a feature, based on the pre-surgical RAW map 422, aortic wall weakness values at the sealing zones. In one or more alternative embodiments, the aortic wall weakness values at the sealing zones may be obtained without using the pre-surgical RAW map.

In one or more embodiments, one or more of the set of pre-EVAR ML models 260 may extract as a feature, for each patient, based on the aortic wall weakness values at the sealing zones, presence of heterogeneous strain at the level of proximal and distal sealing region of the aneurysm, i.e., the neck and iliac arteries.

In one or more embodiments, where the generation procedure 420 has not determined geometric features 425, one or more of the set of pre-EVAR ML models 260 may extract as geometric features 425, for each patient, a deformation value at the aortic neck, and an angle of the aortic neck (i.e. landing site with respect to the aneurysmal dilatation), a tortuosity of the lumen centerline, the asymmetry of the aneurysmal sac, a deformation value at the aortic neck). In one or more embodiments, the feature indicative of a deformation value at the aortic neck and the angle of the aortic neck are determined using the multiphase stack.

In one or more embodiments, one or more of the set of pre-EVAR ML models 260 may extract as a feature, for each patient, a presence of calcifications at the sealing and/or landing site. In one or more embodiments, the feature indicative of presence of calcifications is in the form of a binary value.

In one or more embodiments, one or more of the set of pre-EVAR ML models 260 may extract as a feature, for each patient, a presence of ILT at the sealing and/or landing site. In one or more embodiments, the presence of ILT at the sealing and/or landing site is in the form of a binary value.

To perform predictions, each of the set of pre-EVAR ML models 260 may use as features one or more of: the aortic wall weakness values at the sealing zones, a presence of heterogeneous strain at the level of proximal and distal sealing region of the aneurysm, a deformation value at the aortic neck, and an angle of the aortic neck, a presence of calcifications at the sealing and/or landing site, a presence of ILT at the sealing and/or landing site.

The stent configuration and placement parameters 434 may include: a location on where to place the stent (i.e., neck, iliac arteries), a size of stent, a type of stent, and a need for a fenestrated stent.

In one or more embodiments, the second pre-EVAR ML model 264 outputs an annotated image showing the optimal configuration and placement of the stent for the given patient.

The pre-surgical EVAR planning procedure 400 outputs an EVAR intervention suitability assessment 432. The EVAR intervention suitability assessment 432 may include the strain map 428, the strain heterogeneity map and the strain/relative deformation map, the regional aortic weakness (RAW) map 422, the ILT map 424, the calcification distribution map 426, deformation values and angle of the aortic neck, and a score indicative of a suitability of the intervention. Additionally, the score may be provided with a potential explanation for the score which has been determined based on the strain, calcification, ILT maps and geometric features (e.g. based on values being within our/outside of ranges and/or above/below thresholds). It will be appreciated that the score and the potential explanation may be provided by the set of pre-EVAR ML models 260 based on the data learned from outcomes of past EVAR interventions.

In one or more alternative embodiments, the pre-surgical EVAR planning procedure 400 may further output optimal stent configuration and positioning parameters 434 for the EVAR intervention based on at least the geometric features.

In one or more embodiments, the pre-surgical EVAR planning procedure 400 transmits the EVAR intervention suitability assessment 432 and/or optimal stent configuration and positioning parameters 434 which causes the EVAR intervention suitability assessment 432 and/or optimal stent configuration and positioning parameters 434 for the EVAR intervention to be displayed on a display interface of an electronic device, such as the workstation computer 215. As a non-limiting example, the EVAR intervention suitability assessment 432 (i.e., the different maps and values) and optimal stent configuration and positioning parameters 434 may be displayed on a user interface and interacted with by a medical professional.

In one or more other embodiments, the pre-surgical EVAR planning procedure 400 stores the EVAR intervention suitability assessment 432 and/or optimal stent configuration and positioning parameters 434 for the EVAR intervention. In one or more alternative embodiments, the pre-surgical EVAR planning procedure 400 transmits the EVAR intervention suitability assessment 432 and/or optimal stent configuration and positioning parameters 434 for the EVAR intervention to another electronic device.

### Post-Surgical EVAR Monitoring Procedure

The post-surgical EVAR monitoring procedure 500 is used to monitor a given patient after an EVAR intervention. For example, the given patient may be a patient for which the pre-surgical EVAR planning procedure 400 has outputted a requirement for an EVAR intervention, and who has undergone the EVAR intervention.

The post-surgical EVAR monitoring procedure 500 uses the set of post-EVAR ML models 270 to inter alia: (i) determine, based on strain maps, an indication of a correct placement and sealing of the stent after the EVAR intervention; (ii) determine, based on strain maps, early presence of excessive deformation of the aortic wall at the location of the EVAR as indicative of pressurization and presence of an endoleak and stratify the risk for re-intervention; and (iii) perform early assessment indicative of stent-graft migration.

The post-surgical EVAR monitoring procedure 500 receives the pre-EVAR multiphase image stack 410, a post-EVAR baseline multiphase image stack 504, and optionally a post-EVAR follow-up multiphase image stack 508.

The pre-EVAR multiphase image stack 410 is the multiphase image stack used during the pre-surgical EVAR planning procedure 400.

The post-EVAR baseline multiphase image stack 504 is a multiphase image stack having been acquired by the medical imaging apparatus 210 at the first post-operative imaging session. The post-EVAR baseline multiphase image stack 504 will be used to determine the post-surgical strain baseline.

The post-EVAR follow-up multiphase image stack 508 is a multiphase stack having been acquired by the medical imaging apparatus 210 after the post-EVAR baseline multiphase image stack 504, at a follow-up imaging session.

In one or more embodiments, the post-surgical EVAR monitoring procedure 500 receives a plurality of post-EVAR follow-up multiphase image stacks having been acquired at subsequent follow-ups (e.g., 3 months, 6 months, 9 months, etc.).

The post-surgical EVAR monitoring procedure 500 executes a generation procedure 520, the generation procedure 520 being similar to the generation procedure 420.

The generation procedure 520 is executed to obtain, based on respectively the pre-EVAR multiphase image stack 410, the post-EVAR baseline multiphase image stack 504, and the post-EVAR follow-up multiphase image stack 508: a pre-EVAR strain map 428, a post-EVAR baseline strain map 524 and a post-EVAR follow-up strain map 528.

In one or more embodiments, the pre-EVAR strain map 428, the post-EVAR baseline strain map 524 and the post-EVAR follow-up strain map 528 may be generated by another computing device and may be received by the post-surgical EVAR monitoring procedure 500.

The pre-EVAR strain map 428, the post-EVAR baseline strain map 524 and the post-EVAR follow-up strain map 528 are provided as an input to the set of post-EVAR ML models 270.

The post-surgical EVAR monitoring procedure 500 uses a first post-EVAR ML model 272 to generate, based on the pre-EVAR strain map 428 and the post-EVAR baseline strain map 524, a third set of features and to determine, based on the third set of features, an indication of a presence of pressurization and correct stent placement 532.

The first post-EVAR ML model 272 determines if there is presence of excessive deformation of the aortic wall at the location of the EVAR for example based on a threshold.

It will be appreciated that that by comparing local strain to identify pressurization, the first post-EVAR ML model 272 evaluates the correct placement of the stent and correct exclusion of the aneurysmal sac from flow.

If there is presence of excessive deformation of the aortic wall at the location of the EVAR, this may be indicative of pressurization and presence of an endoleak.

If there is no presence of excessive deformation of the aortic wall at the location of the EVAR, this may indicate a lack of blood flow between the stent-graft and the aortic wall suggesting an ideal isolation of the aneurysmal sac.

The post-surgical EVAR monitoring procedure 500 uses the second post-EVAR ML model 274 to generate, based on the pre-EVAR strain map 428, the post-EVAR baseline strain map 524 and the post-EVAR follow-up strain map 528, a fourth set of features and to determine, based on the fourth set of features, a presence and type of endoleak 534 and a risk for re-intervention.

To stratify the risk for re-intervention, the post-surgical EVAR monitoring procedure 500 identifies low-risk re-intervention and high-risk re-intervention by: (i) predicting, using the second post-EVAR ML model 274, based on the post-EVAR baseline strain map 524 and the post-EVAR follow-up strain map 528, changes in the areas of repaired aneurysm based on the strain evolution; and (ii) detecting, using the third post-EVAR ML model 276, presence of an endoleak and endoleak type 534.

The post-surgical EVAR monitoring procedure 500 uses the third post-EVAR ML model 276 to generate, based on the pre-EVAR strain map 428, the post-EVAR baseline strain map 524 and the post-EVAR follow-up strain map 528, a fifth set of features, and assess, based on the fifth set of features, an indication of stent-graft migration 538.

In one or more embodiments, the third post-EVAR ML model 276 assesses the displacement of the aortic wall in a 3D map, which is indicative of a pressurization of the space between the EVAR and the aortic wall, which may be indicative of a presence of an endoleak.

It will be appreciated that the output of first post-EVAR ML model 272, the second post-EVAR ML model 274 and the third post-EVAR ML model 276 may be provided by less or more ML models and their functionality may combined and/or split.

In one or more embodiments, the first post-EVAR ML model 272 is used in combination with another ML model such as the third post-EVAR ML model 276 which has been trained to perform visual object recognition in strain maps to determine and assess the displacement of the aortic wall in a 3D map.

It will be appreciated that the first post-EVAR ML model 272 has been trained to predict changes in area of repaired aneurysms based on strain maps of patients having undergone EVAR interventions, and that the second post-EVAR ML model 274 has been trained to detect types of endoleaks based on strain maps and multiphase stacks of patients having undergone EVAR interventions.

In one or more embodiments, the post-surgical EVAR monitoring procedure 500 performs early assessment of stent-graft migration based on the post-EVAR follow-up strain map 528.

In the presence of an endoleak, clinicians plan the type of treatment based on the endoleak type and size of the aneurysm sac. From a clinical point of view, endoleaks are classified into five different types:

Type 1 can occur at the proximal end or distal end of the graft attachment area of the artery when blood flow leaks into the aneurysm sac. Type I is generally most common after repair of aortic aneurysms.

Type 2 occurs when the retrograde flow through the branches fills the aneurysm sac (e.g. lumbar or inferior mesenteric artery). It is the most common endoleak type which may resolve spontaneously. But in some cases, can result in embolization of the branch vessel if the aneurysm continuously expands in size.

Type 3 is the result of the mechanical failure of the stent-graft including fracture of the stent-graft, hole on the graft fabric junctional separation of the graft components.

Type 4 can occur when blood leaks across the graft due to its porosity.

Type 5 is the continuous expansion of the aneurysm sac which is not a true leak (endotension).

The post-surgical EVAR monitoring procedure 500 outputs a pressurization identification and correct stent placement 532, a presence and type of endoleak 534 with risk for re-intervention, and assessment of stent-graft migration 538.

It will be appreciated that the ability to assess individual aortas based on their specific level of deformation, as indicative of pressurization, provides valuable information for clinical assessment and will result in an improved and patient-specific management. Additionally, post-surgical risk stratification has the potential to reduce CT imaging follow-ups frequency and, consequently, radiation exposure for patients at low risk for complications as well as overall healthcare costs.

Having explained the pre-surgical EVAR planning procedure 400 and the post-surgical EVAR monitoring procedure 500, the pre-surgical EVAR planning training procedure 600 and the post-surgical EVAR monitoring training procedure 700 will now be described in accordance with one or more non-limiting embodiments of the present technology.

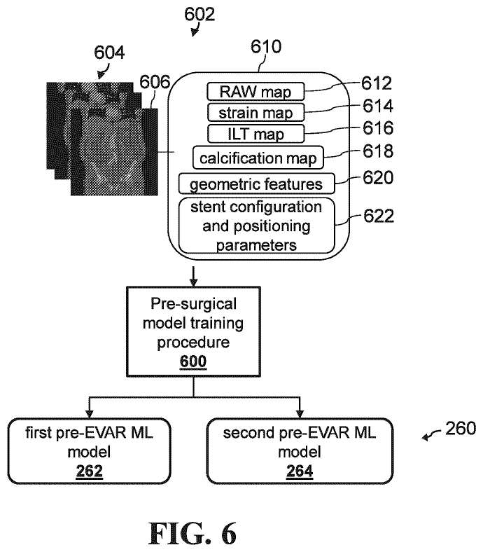

With reference to FIG. 6, there is illustrated the pre-surgical EVAR planning training procedure 600 in accordance with one or more non-limiting embodiments of the present technology.

### Pre-Surgical EVAR Planning Training Procedure

The pre-surgical EVAR planning training procedure 600 is executed by the server 230. In one or more other embodiments, the pre-surgical EVAR planning training procedure 600 may be executed by another electronic device such as the workstation computer 215. In one or more alternative embodiments, at least a portion of the pre-surgical EVAR planning training procedure 600 may be executed by the server 230 and another portion may be executed by another electronic device.

The pre-surgical EVAR planning training procedure 600 aims at training the set of pre-EVAR ML models 260 to determine optimal values of parameters (i.e. ranges and/or thresholds) for predicting procedure outcomes in order to classify EVAR suitability, and predict the optimal stent configuration and placement for a reduced likelihood of post-procedural complications. More specifically, during the pre-surgical EVAR planning training procedure 600, the pre-EVAR ML models 260 are trained based on one or more of: RAW map 612, the strain map 614 including strain heterogeneity map and strain/relative deformation map, the ILT map 616, the calcification distribution map 618, the geometric features 620, as well as stent configuration and positioning parameters 622.

In one or more embodiments, a plurality of ML models may be trained based on different combinations of data including the RAW map 612, the strain map 614 including strain heterogeneity map and strain/relative deformation map, the ILT map 616, the calcification distribution map 618, the geometric features 620, as well as stent configuration and positioning parameters 622 to extract features and to determine an optimal parameter which is indicative of a suitability and/or success of performing an EVAR intervention.

As a non-limiting example, a first ML model may be trained based on the RAW map 612 and strain heterogeneity map, while another ML model may be trained based on the RAW map 612, the strain map 614 including strain heterogeneity map and strain/relative deformation map, the ILT map 616, the calcification distribution map 618, the geometric features 620, as well as stent configuration and positioning parameters 622 and their performance may be compared to determine which model provides the most accurate prediction.

Thus, a given ML model may learn to weigh each of the regional aortic weakness (RAW) map, strain heterogeneity map, strain/relative deformation map, intraluminal thrombus thickness (ILT) map, the calcification distribution map and aortic neck angle to obtain a parameter that is the best indicator of EVAR success.

The pre-EVAR ML models 260 are trained in a supervised manner by using as a label positive (i.e. successful) outcomes of EVAR on patients as well as negative (i.e. unsuccessful) outcomes of EVAR on patients. For each patient of a set of patients, medical images in the form of multiphase stacks are acquired at corresponding time periods prior to the EVAR intervention and are labelled by clinicians as having a positive or negative outcome based on post-surgical assessments. It will be appreciated that ideally the images may be acquired using the same medical imaging apparatus such as the medical imaging apparatus 210 so as to reduce artefacts and machine-specific differences.

In one or more embodiments, the second pre-EVAR ML model 264 is trained to determine optimal stent placement and configuration based on one or more of: the RAW map 612, the strain map 614 including strain heterogeneity map and strain/relative deformation map, the ILT map 616, the calcification distribution map 618, the geometric features 620, as well as stent configuration and positioning parameters 622.

The stent configuration and positioning parameters 622 may include, for each patient, a location where the stent was placed (e.g., neck, iliac arteries), a size of stent, a type of stent, and a need for a fenestrated stent.

In one or more embodiments, the pre-surgical EVAR planning training procedure 600 may generate, based on medical images in the form of multiphase stacks, one or more of the RAW map 612, the strain map 614 including strain heterogeneity map and strain/relative deformation map, the ILT map 616, the calcification distribution map 618, the geometric features 620, as well as stent configuration and positioning parameters 622.

In one or more other embodiments, one or more of the RAW map 612, the strain map 614 including strain heterogeneity map and strain/relative deformation map, the ILT map 616, the calcification distribution map 618, the geometric features 620, as well as stent configuration and positioning parameters 622 may be generated by one or more other electronic devices and then received by the pre-surgical EVAR planning training procedure 600.

The pre-surgical EVAR planning training procedure 600 thus obtains the pre-surgical EVAR planning training dataset 602. The pre-surgical EVAR planning training dataset 602 thus comprises patients for which the EVAR intervention resulted in a positive outcome as wall as patients for which the EVAR intervention resulted in a negative outcome.

The pre-surgical EVAR planning training dataset 602 comprises a plurality of training examples 604 each representing a patient, where a given training example 606 representing a given patient is associated with the RAW map 612, the strain map 614 including strain heterogeneity map and strain/relative deformation map, the ILT map 616, the calcification distribution map 618, the geometric features 620, as well as stent configuration and positioning parameters 622

The pre-surgical EVAR planning training procedure 600 accesses the set of pre-EVAR ML models 260. In one or more embodiments, the pre-surgical EVAR planning training procedure 600 initializes model parameters and hyperparameters of each of the set of pre-EVAR ML models 260.

### Pre-Surgical Model Training

The pre-surgical EVAR planning training procedure 600 begins training each of the set of pre-EVAR ML models 260 based on the pre-surgical EVAR planning training dataset 602.

It will be appreciated that the training of the first pre-EVAR ML model 262 and the second pre-EVAR ML model 264 may be performed at different moments in time or may be performed in parallel.

During training, the pre-surgical EVAR planning training procedure 600 uses each of the set of pre-EVAR ML models 260 to extract features and perform predictions based on the pre-surgical EVAR planning training dataset 602.

In one or more embodiments, one or more of the set of pre-EVAR ML models 260 may extract as a feature, based on the pre-surgical RAW map 612, aortic wall weakness values at the sealing zones. In one or more alternative embodiments, the aortic wall weakness values at the sealing zones may be obtained without using the pre-surgical RAW map.

In one or more embodiments, one or more of the set of pre-EVAR ML models 260 may extract as a feature, for each patient, based on the aortic wall weakness values at the sealing zones, a feature indicative of presence of heterogeneous strain at the level of proximal and distal sealing region of the aneurysm, i.e. the neck and iliac arteries.

In one or more embodiments, one or more of the set of pre-EVAR ML models 260 may extract as a feature, for each patient, a deformation value at the aortic neck, and an angle of the aortic neck (i.e. landing site with respect to the aneurysmal dilatation). In one or more embodiments, the deformation value at the aortic neck and the angle of the aortic neck are determined using the multiphase stack.

In one or more embodiments, one or more of the set of pre-EVAR ML models 260 may extract as a feature, for each patient, a presence of calcifications at the sealing and/or landing site. In one or more embodiments, the presence of calcifications is in the form of a binary value.

In one or more embodiments, one or more of the set of pre-EVAR ML models 260 may extract as a feature, for each patient, a presence of ILT at the sealing and/or landing site. In one or more embodiments, the presence of ILT at the sealing and/or landing site is in the form of a binary value.

To perform predictions, each of the set of pre-EVAR ML models 260 may use as features one or more of: the aortic wall weakness values at the sealing zones, a presence of heterogeneous strain at the level of proximal and distal sealing region of the aneurysm, a deformation value at the aortic neck, and an angle of the aortic neck, a presence of calcifications at the sealing and/or landing site, a presence of ILT at the sealing and/or landing site.

It will be appreciated that that the first pre-EVAR ML model 262 is trained with the respective EVAR outcome as a target, and the second pre-EVAR ML model 264 is trained with the stent configuration and positioning parameters 622 as a target.

The pre-surgical EVAR planning training procedure 600 uses a respective loss function, for each of the set of pre-EVAR ML models 260, to determine a loss based on the respective prediction and the respective labels.

The pre-surgical EVAR planning training procedure 600 then updates respectively, based on the determined loss, the model parameters of the respective one of the set of pre-EVAR ML models 260.

The pre-surgical EVAR planning training procedure 600 determines if a respective termination condition is reached or satisfied for a respective one of the set of pre-EVAR ML models 260. The termination condition may include one or more of: convergence of the model, a desired accuracy, a computing budget, a maximum training duration, a lack of improvement in performance, a system failure, and the like.

If the respective termination condition is not satisfied, the pre-surgical EVAR planning training procedure 600 continues the training of the respective one of the set of pre-EVAR ML models 260 by iterating over the pre-surgical training dataset until the termination condition is satisfied.

If the respective termination condition is satisfied, the pre-surgical EVAR planning training procedure 600 outputs the respective one of the trained set of pre-EVAR ML models 260.

The pre-surgical EVAR planning training procedure 600 may then perform a validation procedure and a testing procedure to validate the performance of the each of the set of pre-EVAR ML models 260 and fine-tune its parameters. The pre-surgical EVAR planning training procedure 600 then outputs the trained first pre-EVAR ML model 262 and the trained second pre-EVAR ML model 264.

The trained first pre-EVAR ML model 262 and the trained second pre-EVAR ML model 264 may then be used respectively to predict, for a new patient, on whether to perform an EVAR procedure and to determine suitability parameters for the placement of an EVAR based on the relative strength and heterogeneity of the aortic tissue.

The trained pre-EVAR ML models 260 may then be stored, for example in the database 235, or may be transmitted over the communication network 220 to another electronic device.

### Post-Surgical EVAR Monitoring Training Procedure

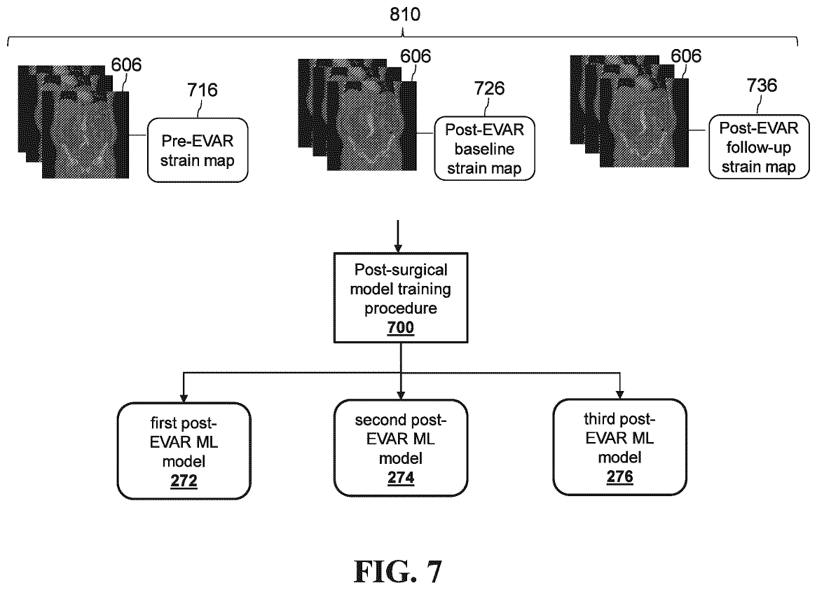

With reference to FIG. 7, the post-surgical EVAR monitoring training procedure 700 will now be explained in accordance with one or more non-limiting embodiments of the present technology.

The post-surgical EVAR monitoring training procedure 700 is executed by the server 230. In one or more other embodiments, the post-surgical EVAR monitoring training procedure 700 may be executed by another electronic device such as the workstation computer 215. In one or more alternative embodiments, at least a portion of the post-surgical EVAR monitoring training procedure 700 may be executed by the server 230 and another portion may be executed by another electronic device.

The post-surgical EVAR monitoring training procedure 700 aims at training the set of post-EVAR ML models 270 to: confirm correct placement and sealing of the stent after the EVAR intervention, assess, based on strain maps, early presence of excessive deformation of the aortic wall at the location of the EVAR as indicative of pressurization and presence of an endoleak and stratify the risk for re-intervention, and perform early assessment of stent-graft migration

To do so, at each of the follow-up scans (1 month, 3 months, etc.) after the EVAR intervention, a strain map is generated and the one or more of the set of post-EVAR ML models 270 is trained to monitor the strain evolution during follow ups and find patterns, based on changes in strain, to identify, or predict, the early presence of endoleak or endotension and stratify the risk for re-intervention.

The post-surgical EVAR monitoring training procedure 700 receives the pre-surgical strain map 716 or pre-EVAR strain map 716.

The post-surgical EVAR monitoring training procedure 700 receives, for each patient, a first post-EVAR strain map 726 or post-EVAR baseline strain map 726. The post-EVAR baseline strain map 726 may be received from the database 235 or from another electronic device. The post-EVAR baseline strain map 726 is a baseline strain map that is indicative of strain values in the aorta after the EVAR intervention for the given patient. It will be appreciated that the first post-EVAR baseline strain map 726 is generated based on multiphase stacks acquired at the same time period after the EVAR intervention for each patient.

In one or more embodiments, the first post-EVAR baseline strain map 726 may be generated based on the post-EVAR baseline multiphase stack.

In one or more embodiments, the post-surgical EVAR monitoring training procedure 700 receives, for each patient, a post-EVAR follow-up strain map 736 or post-surgical follow-up strain map 736.

The post-EVAR follow-up strain map 736 is a follow-up strain map that is indicative of strain values in the aorta after the EVAR intervention for the given patient and after the first post-EVAR strain map has been generated.

In one or more embodiments, the post-surgical EVAR monitoring training procedure 700 may receive subsequent follow-up strain maps.

In one or more embodiments, the post-EVAR baseline strain map 726 may be generated based on the post-EVAR follow-up multiphase stack.

In one or more embodiments, the post-surgical EVAR monitoring training procedure 700 generates the post-EVAR training dataset 810. In one or more other embodiments, the post-EVAR training dataset 810 may have been previously generated by another computing device and may be received by the post-surgical EVAR monitoring training procedure 700.

The post-EVAR training dataset 810 comprises, for each patient, an indication of presence of pressurization and correct stent placement, a presence of endoleak and endoleak type, and an assessment of stent migration, which will be respectively used as a target by the first post-EVAR ML model 272, the second post-EVAR ML model 274, and the third post-EVAR ML model 276.

In one or more embodiments, a first post-EVAR ML model 272 is trained to evaluate, based on strain maps, the placement and sealing of the stent and assess the early presence of excessive deformation of the aortic wall at the location of the EVAR as indicative of pressurization.

In one or more embodiments, a second post-EVAR ML model 274 is trained to determine, based on features representative of the strain evolution in strain maps, the early presence of endoleak or endotension. The second post-EVAR ML model 274 is trained to monitor the changes of the strain analysis measurements by extracting features in the follow up images and find patterns based on the changes that are indicative of a presence and type of endoleak, and a risk for re-intervention.

In one or more embodiments, the second post-EVAR ML model 274 is trained to identify endoleaks in images and based on strain maps and to classify them by type.

A third post-EVAR ML model 276 is trained to determine, based on a set of features indicative of strain evolution, an indication of early stent-graft migration.

The post-surgical EVAR monitoring training procedure 700 uses a respective loss function for each of the set of post-EVAR ML models 270 to determine a loss based on the respective prediction and the respective labels or targets.

The post-surgical EVAR monitoring training procedure 700 then updates, based on the determined loss, the model parameters of each of the set of post-EVAR ML models 270.

The post-surgical EVAR monitoring training procedure 700 determines if a respective termination condition is reached or satisfied for each of the set of post-EVAR ML models 270. The termination condition may include one or more of: convergence of the model, a desired accuracy, a computing budget, a maximum training duration, a lack of improvement in performance, a system failure, and the like.

If the respective termination condition is not satisfied, the post-surgical EVAR monitoring training procedure 700 continues the training of the respective one of the set of post-EVAR ML models 270 by iterating over the post-EVAR training dataset 810 until the termination condition is satisfied.

If the respective termination condition is satisfied, the post-surgical EVAR monitoring training procedure 700 outputs the respective one of the trained post-EVAR ML models 270.

The post-surgical EVAR monitoring training procedure 700 may then perform a validation procedure and a testing procedure to validate the performance of the each of the set of post-EVAR ML models 270 and fine-tune its parameters. The post-surgical EVAR monitoring training procedure 700 then outputs the trained first post-EVAR ML model 272, the trained second post-EVAR ML model 274, and the trained third post-EVAR ML model 276.

The trained post-EVAR ML models 270 may then be stored, for example in the database 235 or may be transmitted over the communication network 220 to another electronic device.

The trained post-EVAR ML models 270 may then be used to: determine correct placement and sealing of the stent after the EVAR intervention, and assess, based on strain maps, early presence of excessive deformation of the aortic wall at the location of the EVAR as indicative of pressurization and presence of an endoleak and stratify the risk for re-intervention, and perform early assessment of stent-graft migration

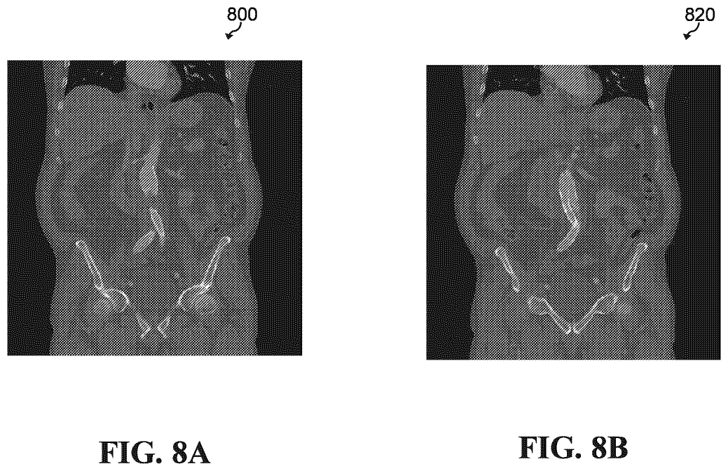

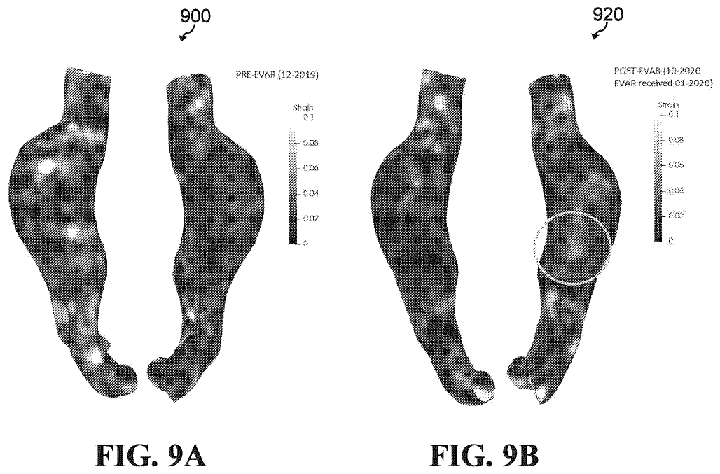

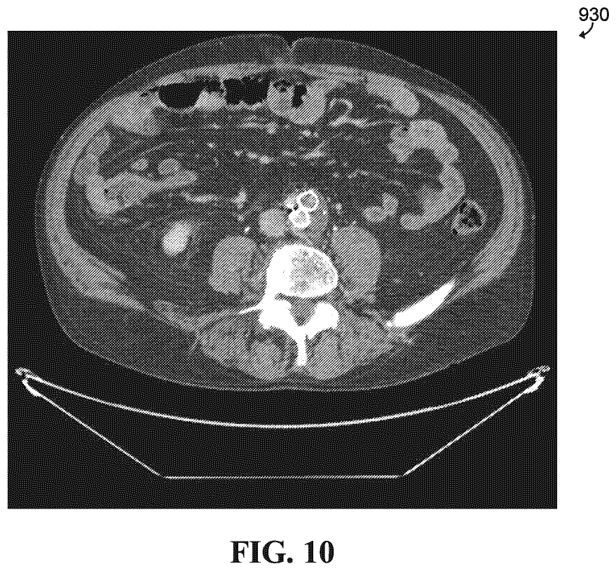

With reference to FIGS. 8A, 8B, 9A, 9B and 10, there are shown respectively coronal view CT scan images 800, 820, a pre-surgical strain map 900 a and post-surgical strain map 920 and an axial view of a CT scan image 930 of an abdominal aortic aneurysm (AAA) in a patient who underwent endovascular aortic aneurysm repair in January 2020 after radiological assessment performed in December 2018 and December 2019. In October 2020 a 10-month post-operative contrast CT scan (FIGS. 9A and 9B) reported as negative for endoleak on follow-up imaging while the aneurysmal sac had not reduced in diameter. A post-EVAR monitoring procedure was performed and the post-surgical strain map demonstrated persistent maximum principal strain at the wall (FIG. 10).

The persistent elevated strain reported by assessment prompted a repeated review of the CT imaging by the radiology team. A small and previously unidentified type 2 lumbar endoleak just above the aortic bifurcation was contributing to sac pressurization (FIG. 10).

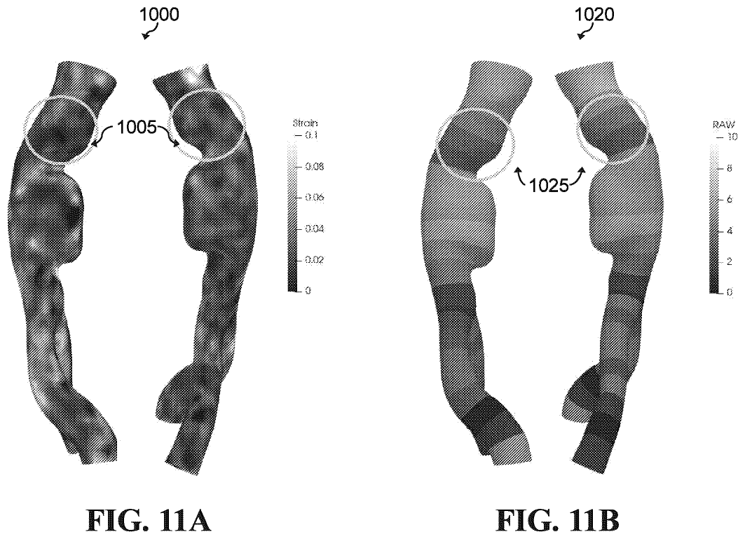

FIG. 11A shows a pre-EVAR strain map 1000 and FIG. 11B shows a pre-surgical sectional RAW map 1020, where zone 1005 shows a low, homogenous strain in the aortic neck region and zone 1025 shows a low RAW index in the aortic neck region (i.e., proximal landing/sealing zone for the stent device during EVAR procedure). At the post-operative follow up the patient showed no endoleaks and a stable aneurysm sac. At a 9-month follow-up the patient showed no signs of endoleaks and a reduced aneurysm sac.

### Method Description

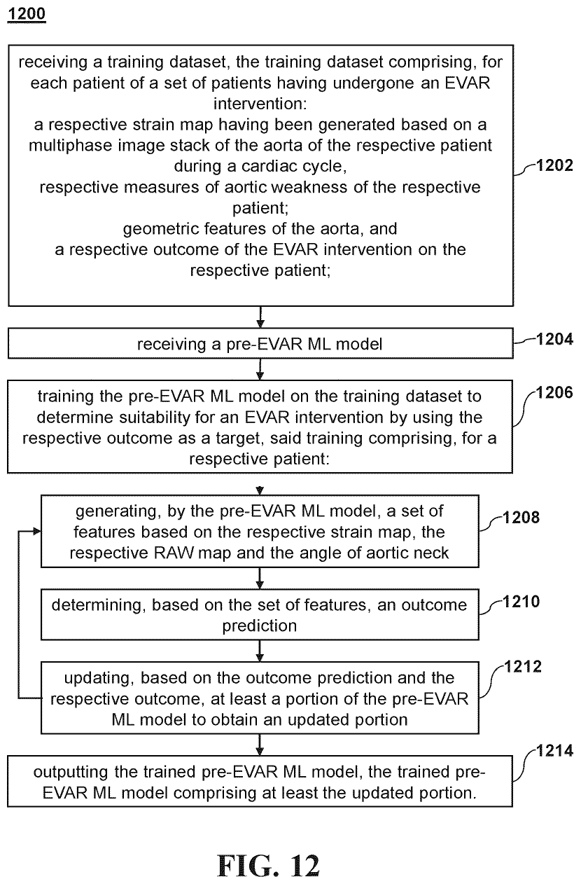

FIG. 12 illustrates a flowchart of a method 1200 for training a machine learning model to determine suitability of a given patient for an endovascular aortic repair (EVAR) intervention, the method being executed in accordance with one or more non-limiting embodiments of the present technology.

In one or more embodiments, the server 230 comprises a processor such as the processor 110 and/or the GPU 111 operatively connected to a non-transitory computer readable storage medium such as the solid-state drive 120 and/or the random-access memory 130 storing computer-readable instructions. The processor, upon executing the computer-readable instructions, is configured to or operable to execute the method 1200.

The method 1200 begins at processing step 1202.

At processing step 1202, the processor receives a training dataset 602, the training dataset 602 comprising, for each patient of a set of patients having undergone an EVAR intervention: a respective strain map 614 having been generated based on a multiphase image stack 410 of the aorta of the respective patient during a cardiac cycle, the regional aortic weakness (RAW) map 612, geometric features of the aorta (e.g., aortic neck angle) 620 and a respective outcome of the EVAR intervention on the respective patient.

In one or more embodiments, each patient is associated with one or more of: a regional aortic weakness (RAW) map 612, a strain map 614, the strain heterogeneity map and the strain/relative deformation map, the ILT map 616, the calcification distribution map 618, geometric features 620, and stent configuration and positioning parameters 622.

In one or more embodiments, measures of aortic weakness in the aorta of the respective patient are provided instead of the complete RAW map 612.

At processing step 1204, the processor receives a pre-EVAR ML model. In one or more embodiments, the processor receives a respective one of the set of pre-EVAR ML models 260 by initializing its respective model parameters and respective hyperparameters.

At processing step 1206, the processor trains the pre-EVAR ML model on the training dataset to determine suitability for an EVAR intervention by using the respective outcome as a target, said training comprising processing steps 1208-1212 which may be repeated on the training dataset.

In one or more embodiments, the first pre-EVAR ML model 262 is trained with the respective EVAR outcome as a target, and the second pre-EVAR ML model 264 is trained with the stent configuration and positioning parameters 622 as a target.

At processing step 1208, the processor generates, using a respective one of the set of pre-EVAR ML models 260, a set of features based on the respective strain map 614, the respective RAW map 612 and the geometric features 620.

In one or more embodiments, one or more of the set of pre-EVAR ML models 260 may extract as a feature, based on the pre-surgical RAW map 612, aortic wall weakness values at the sealing zones. In one or more alternative embodiments, the aortic wall weakness values at the sealing zones may be obtained without using the pre-surgical RAW map.

In one or more embodiments, one or more of the set of pre-EVAR ML models 260 may extract as a feature, for each patient, based on the aortic wall weakness values at the sealing zones, presence of heterogeneous strain at the level of proximal and distal sealing region of the aneurysm, i.e. the neck and iliac arteries.

In one or more embodiments, one or more of the set of pre-EVAR ML models 260 may extract as a feature, for each patient, a deformation value at the aortic neck, and an angle of the aortic neck (i.e. landing site with respect to the aneurysmal dilatation). In one or more embodiments, the deformation value at the aortic neck and the angle of the aortic neck are determined using the multiphase stack.

In one or more embodiments, one or more of the set of pre-EVAR ML models 260 may extract as a feature, for each patient, a presence of calcifications at the sealing and/or landing site. In one or more embodiments, the presence of calcifications is in the form of a binary value.

In one or more embodiments, one or more of the set of pre-EVAR ML models 260 may extract as a feature, for each patient, a presence of ILT at the sealing and/or landing site. In one or more embodiments, the presence of ILT at the sealing and/or landing site is in the form of a binary value.

At processing step 1120, the processor determines, using a respective one of the set of pre-EVAR ML models 260, based on the set of features, an outcome prediction.

At processing step 1212, the processor updates, based on the outcome prediction and the respective outcome, at least a portion of the pre-EVAR ML model to obtain an updated portion.

In one or more embodiments, the processor uses a respective loss function, for each of the set of pre-EVAR ML models 260, to determine a loss based on the respective prediction and the respective labels. The processor then updates respectively, based on the determined loss, the model parameters of each of the set of pre-EVAR ML models 260.

At processing step 1214, the processor outputs the trained pre-EVAR ML model, the trained pre-EVAR ML model comprising at least the updated portion.

In one or more embodiments, the processor determines if a respective termination condition is reached or satisfied for a respective one of the set of pre-EVAR ML models 260. The respective termination condition may include one or more of: convergence of the model, a desired accuracy, a computing budget, a maximum training duration, a lack of improvement in performance, a system failure, and the like.

The processor then outputs one or more of the trained first pre-EVAR ML model 262 and the trained second pre-EVAR ML model 264.

The trained first pre-EVAR ML model 262 and the trained second pre-EVAR ML model 264 may then be used respectively to predict, for a new patient, on whether to perform an EVAR procedure and to determine the best placement of an EVAR as well as establishing suitability parameters for the placement of an EVAR based on the relative strength and heterogeneity of the aortic tissue.

The method 1200 then ends.

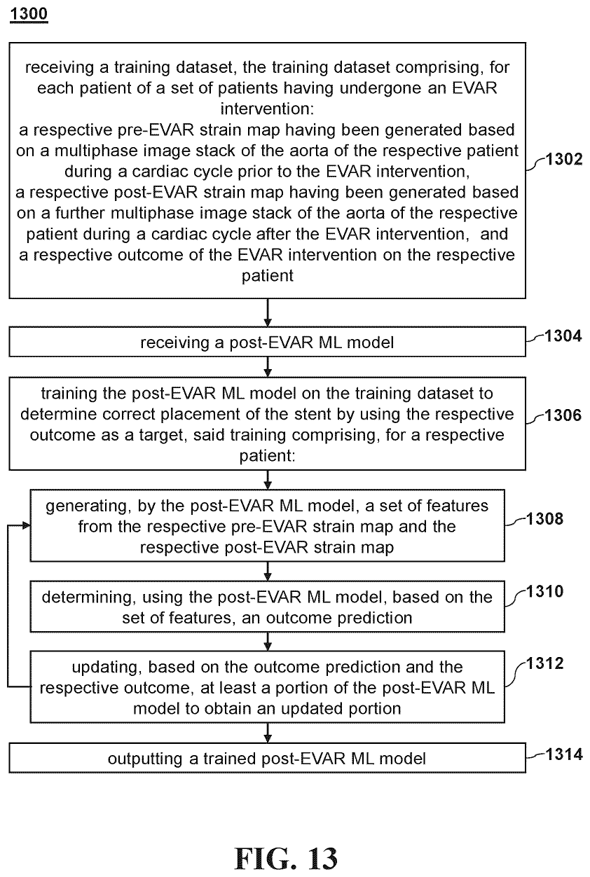

FIG. 13 depicts a flowchart of a method 1300 for training a machine learning (ML) model to monitor a given patient after an endovascular aortic repair (EVAR) intervention in accordance with one or more non-limiting embodiments of the present technology.

In one or more embodiments, the server 230 comprises a processor such as the processor 110 and/or the GPU 111 operatively connected to a non-transitory computer readable storage medium such as the solid-state drive 120 and/or the random-access memory 130 storing computer-readable instructions. The processor, upon executing the computer-readable instructions, is configured to or operable to execute the method 1300.

The method 1300 begins at processing step 1302.

At processing step 1302, the processor receives a training dataset 810, the training dataset 810 comprising, for each patient of a set of patients having undergone an EVAR intervention: a respective pre-EVAR strain map 716 having been generated based on a multiphase image stack of the aorta of the respective patient during a cardiac cycle prior to the EVAR intervention, a respective post-EVAR baseline strain map 726 having been generated based on a further multiphase image stack of the aorta of the respective patient during a cardiac cycle after the EVAR intervention, and a respective outcome of the EVAR intervention on the respective patient.

In one or more embodiments, the processor receives, for each patient, a post-EVAR follow-up strain map 736.

At processing step 1304, the processor receives a post-EVAR ML model. In one or more embodiments, the processors receives a respective one of the set of post-EVAR ML models 270 by initializing the respective one of the set of post-EVAR ML model based on model parameters and hyperparameters.

At processing step 1306, the processor trains a respective one of the post-EVAR ML models 270 on the training dataset 810 to determine correct placement of the stent by using the respective outcome as a target, said training comprising processing steps 1308-1212 which may be repeated on the training dataset.

In one or more embodiments, the training dataset 810 comprises, for each patient, an indication of presence of pressurization and correct stent placement, a presence of endoleak and endoleak type, and an assessment of stent migration, which will be respectively used as a target by the first post-EVAR ML model 272, the second post-EVAR ML model 274, and the third post-EVAR ML model 276.

At processing step 1308, the processor generates, using a respective one of the set of post-EVAR ML models 270, a set of features from the pre-EVAR strain map 716, the post-EVAR baseline strain map 726, and the post-EVAR follow-up strain map 736.

At processing step 1310, the processor determines using a respective one of the set of post-EVAR ML models 270, based on the set of features, an outcome prediction.

At processing step 1312, the processor updates based on the outcome prediction and the respective outcome, at least a portion of the post-EVAR ML model to obtain an updated portion.

The processor uses a loss function to determine a loss based on the respective prediction and the respective labels or targets. The processor then updates, based on the determined loss, the model parameters of the respective one of the set of post-EVAR ML models 270.

In one or more embodiments, a first post-EVAR ML model 272 is trained to evaluate, based on strain maps, the placement and sealing of the stent and assess the early presence of excessive deformation of the aortic wall at the location of the EVAR.

In one or more embodiments, a second post-EVAR ML model 274 is trained to determine, based on features representative of the strain evolution in strain maps, the early presence of endoleak or endotension. The second post-EVAR ML model 274 is trained to monitor the changes of the strain analysis measurements by extracting features during the follow ups and find patterns based on the changes that are indicative of a presence and type of endoleak, and a risk for re-intervention.

In one or more embodiments, the second post-EVAR ML model 274 is trained to identify endoleaks in images and based on strain maps and to classify them by type.

The third post-EVAR ML model 276 is trained to assess, based on a set of features indicative of strain evolution, early stent-graft migration.

At processing step 1314, the processor outputs the trained post-EVAR ML model, the trained post-EVAR ML model comprising at least the updated portion.

The processor determines if a respective termination condition is reached or satisfied for each of the set of post-EVAR ML models 270. The termination condition may include one or more of: convergence of the model, a desired accuracy, a computing budget, a maximum training duration, a lack of improvement in performance, a system failure, and the like.

If the respective termination condition is not satisfied, the processing continues the training of the respective one of the set of post-EVAR ML models 270 by iterating over the post-EVAR training dataset 810 by repeating processing steps 1308-1312 until the termination condition is satisfied.

If the respective termination condition is satisfied, the processor outputs the respective one of the trained post-EVAR ML models 270.

The trained post-EVAR ML models 270 may then be used to: determine correct placement and sealing of the stent after the EVAR intervention, assess, based on strain maps, early presence of excessive deformation of the aortic wall at the location of the EVAR as indicative of pressurization and presence of an endoleak and stratify the risk for re-intervention, and perform early assessment of stent-graft migration.

The method 1300 then ends.

It should be expressly understood that not all technical effects mentioned herein need to be enjoyed in each and every embodiment of the present technology. For example, embodiments of the present technology may be implemented without the user enjoying some of these technical effects, while other non-limiting embodiments may be implemented with the user enjoying other technical effects or none at all.

Some of these steps and signal sending-receiving are well known in the art and, as such, have been omitted in certain portions of this description for the sake of simplicity. The signals can be sent-received using optical means (such as a fiber-optic connection), electronic means (such as using wired or wireless connection), and mechanical means (such as pressure-based, temperature based or any other suitable physical parameter based).

Modifications and improvements to the above-described implementations of the present technology may become apparent to those skilled in the art. The foregoing description is intended to be exemplary rather than limiting.

### REFERENCES

[1] Ashton H A, Gao L, Kim L G, Druce P S, Thompson S G, Scott R A. “Fifteen-year follow-up of a randomized clinical trial of ultrasonographic screening for abdominal aortic aneurysms”. Br J Surg 94.6 (2007): 696-701.
[2] Assar A N, Zarins C K. “Ruptured abdominal aortic aneurysm: a surgical emergency with many clinical presentations”. Postgrad Med J 85.1003 (2009): 268-73.
[3] Geroulakos G, Nicolaides A. “Infrarenal abdominal aortic aneurysms less than five centimeters in diameter: the surgeon's dilemma”. Eur J Vase Surg 6.6 (1992): 616-22.
[4] Powell J T, Gotensparre S M, Sweeting M J, Brown L C, Fowkes F G, Thompson S G. “Rupture rates of small abdominal aortic aneurysms: a systematic review of the literature”. Eur J Vase Endovasc Surg 41.1 (2011): 2-10.
[5] Cochrane Database Syst Rev, 2014.
[6] Brekken R, Dahl T, Hernes T A N, & Myhre H O (2008). “Reduced strain in abdominal aortic aneurysms after endovascular repair”. Journal of Endovascular Therapy, 15(4), 453-461 https://doi.org/10.1583/07-2349.1.
[7] EVARplanning—Endovascular stent graft configuration and rendering. 2021 EVARplanning. https://www.evarplanning.com/
[8] Desender L, Van Herzeele I, Lachat M, Duchateau J, Bicknell C, Teijink J, Heyligers J, Vermassen F, Desender L, Janssens M, Maertens H, Moreels N, Spriet E, Van Herzeele I, Vermassen F, Chaykovska L, Glenck M, Lachat M, Mayer D, Puipe G, Rancic Z, Buttiens J, Duchateau J, Tielemans Y, Heyligers J, Lijkwan M, Vriens P, Bicknell C, Gibbs R, Hamady M, Jenkins M, Lear R, Riga C, Rudarakanchana N, Thomas R, Bendermacher B, Teijink J, Van Sambeek M. “A multicentre trial of patient specific rehearsal prior to EVAR: impact on procedural planning and team performance”. Eur J Vase Endovasc Surg 53 (3). 2017:354-361.
[9] Sobocinski J, Chenorhokian H, Maurel B, Midulla M, Hertault A, Le Roux M, Azzaoui R, Haulon S. The benefits of EVAR planning using a 3D workstation. Eur J Vase Endovasc Surg. 2013 October; 46(4):418-23. doi: 10.1016/j.ejvs.2013.07.018.
[10] Attallah O, Ma X. “Bayesian neural network approach for determining the risk of re-intervention after endovascular aortic aneurysm repair”. Proc Inst Mech Eng H 228.9 (2014): 857-66.

1. A method for training a machine learning (ML) model to determine suitability of a given patient for an endovascular aortic repair (EVAR) intervention, said method being executed by at least one processor, said method comprising:
receiving a training dataset, the training dataset comprising, for each patient of a set of patients having undergone an EVAR intervention:
a respective strain map having been generated based on a multiphase image stack of the aorta of the respective patient during a cardiac cycle,
respective measures of aortic weakness in the aorta of the respective patient,
geometric features of the aorta of the respective patient; and
a respective outcome of the EVAR intervention on the respective patient;
receiving a pre-EVAR ML model;
training the pre-EVAR ML model on the training dataset to determine suitability for an EVAR intervention by using the respective outcome as a target, said training comprising, for a respective patient:
generating, using the pre-EVAR ML model, a set of features based on the respective strain map, the respective measures of aortic weakness in the aorta of the respective patient and the geometric features of the aorta of the respective patient;
determining, using the pre-EVAR ML model, based on the set of features, an outcome prediction; and
updating, based on the outcome prediction and the respective outcome, at least a portion of the pre-EVAR ML model to obtain an updated portion; and
outputting the trained pre-EVAR ML model, the trained pre-EVAR ML model comprising at least the updated portion.

2. The method of claim 1, wherein
the training dataset further comprises, for each patient of the set of patients having undergone the EVAR intervention, at least one of: a respective calcification distribution map and an intraluminal thrombus thickness (ILT) map; and wherein
said generating the set of features comprises generating features from the at least one of the respective calcification distribution map and the ILT map.

3. (canceled)

4. (canceled)

5. The method of claim 1, wherein the set of features comprises features indicative of: heterogeneous strain at proximal and distal sealing regions of an aneurysm, and a deformation level at the neck.

6. The method of claim 1, wherein the geometric features comprise at least one of: an aortic neck angle, a tortuosity of the lumen centerline, an asymmetry of an aneurysmal sac, and a deformation value at the neck.

7. (canceled)

8. The method of claim 1, wherein
the training dataset further comprises, for each patient of the set of patients having undergone the EVAR intervention: respective dimensions and configuration of a respective stent installed during the EVAR intervention; and wherein
said method further comprises:
receiving a further ML model;
training the further ML model on the training dataset to determine dimensions and configurations of a stent by using the respective dimensions and configuration of a respective stent as a target, said training comprising, for a respective patient:
generating, by the further ML model, a further set of features from the respective strain map, the respective measures of aortic weakness in the aorta of the respective patient and the geometric features;
determining, based on the set of features, a predicted dimension and configuration; and
updating, based on the predicted dimension and configuration and the respective dimensions and configuration, at least a portion of the further ML model to obtain an updated portion; and
outputting the trained further ML model, the trained further ML model comprising at least the updated portion.

9. A method for training a machine learning (ML) model to monitor a given patient after an endovascular aortic repair (EVAR) intervention, the method being executed by at least one processor, said method comprising:
receiving a training dataset, the training dataset comprising, for each patient of a set of patients having undergone an EVAR intervention:
a respective pre-EVAR strain map having been generated based on a multiphase image stack of the aorta of the respective patient during a cardiac cycle prior to the EVAR intervention,
a respective post-EVAR strain map having been generated based on a further multiphase image stack of the aorta of the respective patient during a cardiac cycle after the EVAR intervention,
a respective outcome of the EVAR intervention on the respective patient;
receiving a post-EVAR ML model;
training the post-EVAR ML model on the training dataset to determine correct placement of the stent by using the respective outcome as a target, said training comprising, for a respective patient:
generating, using the post-EVAR ML model, a set of features from the respective pre-EVAR strain map and the respective post-EVAR strain map;
determining, using the post-EVAR ML model, based on the set of features, an outcome prediction; and
updating, based on the outcome prediction and the respective outcome, at least a portion of the post-EVAR ML model to obtain an updated portion; and
outputting the trained post-EVAR ML model, the trained post-EVAR ML model comprising at least the updated portion.

10. (canceled)

11. The method of claim 9, wherein said determining, by the post-EVAR ML model based on the first and the second set of features, the outcome prediction comprises:
identifying, by the post-EVAR ML model, a respective level of pressurization between the respective stent and a respective aortic wall; and
determining, based on the respective level of pressurization being above a threshold, the outcome prediction as being an incorrect sealing.

12. The method of claim 11, further comprising determining based on the respective level of pressurization being below the threshold, the outcome prediction as being a correct sealing.

13. The method of claim 9, wherein the training dataset further comprises, for each patient of the set of patients having undergone the EVAR intervention:
a respective follow-up post-EVAR strain map having been generated based on a follow-up multiphase image stack of the aorta of the respective patient during a cardiac cycle after the respective post-EVAR strain map, and
an indication of a respective presence of an endoleak and an endoleak type; and wherein
said method further comprises:
receiving a further post-EVAR ML model;
training the further post-EVAR ML model on the training dataset to identify and classify endoleaks based on the respective indication of the respective presence of the endoleak and the endoleak type, said training comprising, for a respective patient:
generating, by the further post-EVAR ML model, a set of features from the respective pre-EVAR strain map, the respective post-EVAR strain map, and the respective follow-up post-EVAR strain map;
determining, by the further post-EVAR ML model, based on the set of features, a predicted endoleak presence and a predicted endoleak type; and
updating, based on the predicted endoleak presence and predicted endoleak type and the respective presence of the endoleak and the endoleak type, at least a portion of the further post-EVAR ML model to obtain an updated portion; and
outputting the trained further post-EVAR ML model, the trained further post-EVAR ML model comprising at least the updated portion.

14. The method of claim 13, further comprising, after said determining, by the further post-EVAR ML model, based on the set of features, the predicted endoleak presence and the predicted endoleak type:
determining a size of the aneurysm sac; and
determining, based on the size of the aneurysm sac and the predicted endoleak presence and the predicted endoleak type, a risk for re-intervention.

15. (canceled)

16. A system for training a machine learning (ML) model to determine suitability of a given patient for an endovascular aortic repair (EVAR) intervention, said system comprising:
at least one processor; and
a non-transitory storage medium operatively connected to the at least one processor, the non-transitory storage medium storing instructions,
the at least one processor, upon executing the instructions, being configured for:
receiving a training dataset, the training dataset comprising, for each patient of a set of patients having undergone an EVAR intervention:
a respective strain map having been generated based on a multiphase image stack of the aorta of the respective patient during a cardiac cycle,
respective measures of aortic weakness in the aorta of the respective patient,
geometric features of the aorta of the respective patient; and
a respective outcome of the EVAR intervention on the respective patient;
receiving a pre-EVAR ML model;
training the pre-EVAR ML model on the training dataset to determine suitability for an EVAR intervention by using the respective outcome as a target, said training comprising, for a respective patient:
generating, using the pre-EVAR ML model, a set of features based on the respective strain map, the respective measures of aortic weakness in the aorta of the respective patient and the geometric features of the aorta of the respective patient;
determining, using the pre-EVAR ML model, based on the set of features, an outcome prediction; and
updating, based on the outcome prediction and the respective outcome, at least a portion of the pre-EVAR ML model to obtain an updated portion; and
outputting the trained pre-EVAR ML model, the trained pre-EVAR ML model comprising at least the updated portion.

17. The system of claim 16, wherein
the training dataset further comprises, for each patient of the set of patients having undergone the EVAR intervention, at least one of: a respective calcification distribution map and an intraluminal thrombus thickness (ILT) map; and wherein
said generating the set of features comprises generating features from the at least one of the respective calcification distribution map and the ILT map.

18. (canceled)

19. (canceled)

20. The system of claim 16, wherein the set of features comprises features indicative of: heterogeneous strain at proximal and distal sealing regions of an aneurysm, and a deformation level at the neck.

21. The system of claim 16, wherein the geometric features comprise at least one of: an aortic neck angle, a tortuosity of the lumen centerline, an asymmetry of an aneurysmal sac, and a deformation value at the neck.

22. (canceled)

23. The system of claim 16, wherein
the training dataset further comprises, for each patient of the set of patients having undergone the EVAR intervention: respective dimensions and configuration of a respective stent installed during the EVAR intervention; and wherein
the at least one processor is further configured for:
receiving a further ML model;
training the further ML model on the training dataset to determine dimensions and configurations of a stent by using the respective dimensions and configuration of a respective stent as a target, said training comprising, for a respective patient:
generating, by the further ML model, a further set of features from the respective strain map, the respective measures of aortic weakness in the aorta of the respective patient and the geometric features;
determining, based on the set of features, a predicted dimension and configuration; and
updating, based on the predicted dimension and configuration and the respective dimensions and configuration, at least a portion of the further ML model to obtain an updated portion; and
outputting the trained further ML model, the trained further ML model comprising at least the updated portion.

24. A system for training a machine learning (ML) model to monitor a given patient after an endovascular aortic repair (EVAR) intervention, said system comprising:
at least one processor; and
a non-transitory storage medium operatively connected to the at least one processor, the non-transitory storage medium storing instructions,
the at least one processor, upon executing the instructions, being configured for:
receiving a training dataset, the training dataset comprising, for each patient of a set of patients having undergone an EVAR intervention:
a respective pre-EVAR strain map having been generated based on a multiphase image stack of the aorta of the respective patient during a cardiac cycle prior to the EVAR intervention,
a respective post-EVAR strain map having been generated based on a further multiphase image stack of the aorta of the respective patient during a cardiac cycle after the EVAR intervention,
a respective outcome of the EVAR intervention on the respective patient;
receiving a post-EVAR ML model;
training the post-EVAR ML model on the training dataset to determine correct placement of the stent by using the respective outcome as a target, said training comprising, for a respective patient:
generating, using the post-EVAR ML model, a set of features from the respective pre-EVAR strain map and the respective post-EVAR strain map;
determining, using the post-EVAR ML model, based on the set of features, an outcome prediction; and
updating, based on the outcome prediction and the respective outcome, at least a portion of the post-EVAR ML model to obtain an updated portion; and
outputting the trained post-EVAR ML model, the trained post-EVAR ML model comprising at least the updated portion.

25. (canceled)

26. The system of claim 24, wherein said determining, by the post-EVAR ML model based on the first and the second set of features, the outcome prediction comprises:
identifying, by the post-EVAR ML model, a respective level of pressurization between the respective stent and a respective aortic wall; and
determining, based on the respective level of pressurization being above a threshold, the outcome prediction as being an incorrect sealing.

27. The system of claim 26, wherein the at least one processor is further configured for determining based on the respective level of pressurization being below the threshold, the outcome prediction as being a correct sealing.

28. The system of claim 24, wherein the training dataset further comprises, for each patient of the set of patients having undergone the EVAR intervention:
a respective follow-up post-EVAR strain map having been generated based on a follow-up multiphase image stack of the aorta of the respective patient during a cardiac cycle after the respective post-EVAR strain map, and
an indication of a respective presence of an endoleak and an endoleak type; and wherein
the at least one processor is further configured for:
receiving a further post-EVAR ML model;
training the further post-EVAR ML model on the training dataset to identify and classify endoleaks based on the respective indication of the respective presence of the endoleak and the endoleak type, said training comprising, for a respective patient:
generating, by the further post-EVAR ML model, a set of features from the respective pre-EVAR strain map, the respective post-EVAR strain map, and the respective follow-up post-EVAR strain map;
determining, by the further post-EVAR ML model, based on the set of features, a predicted endoleak presence and a predicted endoleak type; and
updating, based on the predicted endoleak presence and predicted endoleak type and the respective presence of the endoleak and the endoleak type, at least a portion of the further post-EVAR ML model to obtain an updated portion; and
outputting the trained further post-EVAR ML model, the trained further post-EVAR ML model comprising at least the updated portion.

29. The system of claim 28, wherein the at least one processor is further configured for, after said determining, by the further post-EVAR ML model, based on the set of features, the predicted endoleak presence and the predicted endoleak type:
determining a size of the aneurysm sac; and
determining, based on the size of the aneurysm sac and the predicted endoleak presence and the predicted endoleak type, a risk for re-intervention.

30. (canceled)
